(19) United States
(12) Patent Application Publication
(10) Pub. No.: US 2012/0211006 A1
(43) Pub. Date: Aug. 23, 2012

(54) Automated Needle Insertion Mechanism

(75) Inventors: Brijesh S. Gill, Houston, TX (US); Raul G. Longoria, Austin, TX (US); Kevin Aroom, Houston, TX (US); Albert A. Espinoza, Baytown, TX (US); Alex Bjelica, Sacaramento, CA (US); Charles S. Cox, JR., Bellaire, TX (US)

(73) Assignee: BOARD OF REGENTS THE UNIVERSITY OF TEXAS SYSTEM, Austin, TX (US)

(21) Appl. No.: 13/392,185

(22) PCT Filed: Aug. 24, 2010
(86) PCT No.: PCT/US10/46507

§ 371 Date: May 8, 2012

Related U.S. Application Data
(60) Provisional application No. 61/236,442, filed on Aug. 24, 2009.

(51) Int. Cl. A61B 17/34; A61M 16/04

(52) U.S. Cl. 128/200.26; 604/164.1

(57) ABSTRACT

A device is provided for automatically inserting a catheter or other medical implement into a patient. An imaging module (100) identifies a selected point of insertion on the patient. A manipulator module (200) positions a catheter or medical implement at the desired position with respect to the selected point of insertion on the patient. A catheter insertion module (300) or implement insertion module (350) inserts the medical instrument into the patient to complete the desired tasks.

### CROSS REFERENCE TO RELATED APPLICATIONS

This application claims the benefit under 35 U.S.C. §119(e) of U.S. Provisional Patent Application No. 61/236,442, filed on Aug. 24, 2009, which is herein incorporated by reference in its entirety.

### STATEMENT REGARDING FEDERALLY SPONSORED RESEARCH OR DEVELOPMENT

This invention was made with U.S. Government support under Grant No. 0700389 awarded by the National Science Foundation. The government has certain rights in the invention.

### FIELD OF THE INVENTION

In austere battlefield environments, acute hemorrhage accounts for 50% of soldier fatalities and is the primary cause of death in 30% of injured soldiers who die from wounds. Current fluid delivery techniques are manual procedures that require highly-skilled surgeons, a commodity not usually available in combat scenarios. This invention relates to an automated mechanism that obtains vascular access of drugs and fluids to soldiers injured in combat via the insertion of a catheter inside the femoral vein. A modular mechanism includes two independent modules. The first subsystem orients the insertion of the catheter in space. The second subsystem inserts the catheter inside the vein.

### BACKGROUND OF THE INVENTION

In the battlefield, the difference between survival and fatality may be drastically influenced by the degree of crucial pre-hospital medical care that can be provided to the soldier. In such austere combat environments, acute hemorrhage accounts for 50% of soldier fatalities and is the primary cause of death in 30% of injured soldiers who die from wounds. In many cases, soldiers wounded in combat do not have immediate access to emergency medical assistance and must wait for hours before medical evacuation becomes an option, particularly in the scattered battle scenarios typical of the conflicts in Iraq and Afghanistan. In order to help reduce such staggering fatality figures, soldiers require the use of an effective, reliable, and quick method for delivering blood and resuscitating fluids in rugged, far-forward battle scenarios.

Currently, the most traditionally-used routes for fluid delivery involve either intravenous (IV) cannulation with flexible catheters or intra-osseous (10) access with rigid intra-osseous needles, but even though such procedures have proven effective and reliable in controlled hospital and pre-hospital environments, their implementation into the battlefield is greatly impaired by several key war-specific factors, including the lack of available trained surgeons, the tactical combat conditions, and the remote and hostile nature of the battlefield environment itself, which make obtaining vascular access difficult, even for the best-trained surgeons. These complicated conditions call for the need for an automated mechanism that is able to obtain vascular access in a fast, efficient, and reliable manner by harnessing the enhanced precision and repeatability robotic systems have over human surgeons.

Current methods for obtaining vascular access are manual procedures that depend entirely on the expertise and dexterity of the surgeon. Of these methods, IV catheterization via the Seldinger technique is the standard-of-practice procedure and its implementation throughout the years has proven to be effective and safe.

Central IV access sites depend on the type of procedure, but typically include the subclavian vein in the chest, the internal jugular vein in the neck or the femoral vein in the groin area. Using the Seldinger method to perform an IV catheterization, the surgeon typically uses external landmarks to pinpoint the target location, including anatomic landmarks as well as feeling for the pulse of nearby arteries, imaging feedback such as ultrasound or fluoroscopy to pinpoint the appropriate target location is used as an adjunct.

The surgeon then inserts a thin-walled hollow needle into the vein, usually at an angle with respect to the surface of the skin. Once vein penetration is verified by checking for hemostatic pressure inside the needle, the surgeon removes the syringe while holding the needle in place and threads a guidewire through the needle and into the vein. At this point, the needle is removed while holding the guidewire in place and a scalpel is used to make a small incision at the penetration site to ease the insertion of the incoming implements. A dilator is then advanced over the guidewire and into the vein in order to open up the insertion path. Next, the dilator is retracted while holding the guidewire in place, and ultimately, a flexible, conical-tipped catheter is introduced through the guidewire and pushed inside the vein. Once the catheter is inside the vein, the guidewire is removed, leaving the catheter in place.

Another method of obtaining vascular access that has resurfaced in recent years as a viable procedure is the intra-osseous (10) route. Using this procedure, a rigid needle is inserted into the sternum or the tibia, the distal tibia and femur to access the circulatory system through the bone marrow.

Even though the 10 route provides a safe and effective method for delivering drugs during cardiopulmonary resuscitation, it also has the potential to cause extravasation of drugs and fluids into soft tissue, fat or bone emboli, and particularly, although rarely, osteomyelitis, and thus is only favored whenever the IV route cannot be rapidly obtained. Furthermore, current practice also recommends that 10 devices should be used only as a temporary procedure and should be removed as soon as the more conventional IV access may be performed.

Currently there are no known mechanisms that allow for fully-autonomous catheter insertions into the femoral vein. The closest work in this subject involves the development of master-slave mechanisms that can be operated by a surgeon guiding the robot via a haptic interface. Fukuda et al. developed a 3 Degrees-of-Freedom (DOF) slave mechanism that can be controlled via joystick to aid surgeons perform intravenous neurosurgery. Jayender, et al. developed a hybrid impedance control (HIC) scheme to help surgeons perform catheter insertions using a 7 DOF Mitsubishi PA 10-7C slave robot. Most of the emphasis in the literature, however, has been dedicated to the development of needle insertion mechanisms for use in laparoscopy, brachytherapy, and neurosurgery. For instance, Taylor at al. fabricated a telerobotic assistant for laparoscopic surgery using a patented approach that relies on the principles of the four-bar linkage with coupled joint motion to orient the needle about a remote center of motion (RCM) located at the insertion point. Kronreif at al. produced a similar RCM mechanism, however, their mechanism utilizes a planar mechanism with one stationary link that holds the needle tip at the insertion point and a moving link that provides the RCM motion of the needle about the insertion point. However, in a realistic battlefield scenario, it is impossible to assure reliable communications for telerobotic insertion of a needle by a remote surgeon.

### SUMMARY OF THE INVENTION

Because of the reasons stated above, current research has focused on IV vascular access and of all the possible IV sites. The femoral vein is selected as a suitable automatic insertion site because this region has a low tissue resistance (mostly skin and fat), is far away from vital organs, and the vein is easily accessible when the patient lies at on his/her back, requiring only a simple landmark-based tactile method of identifying the target vein. In one embodiment, the invention successfully introduces a cannula into a major blood vessel with no human intervention, with the subject lying in a supine position within the range of motion for the device.

The inventive mechanism automatically inserts a catheter into the femoral vein. Although some preparation by the field medic is allowed, the device in one embodiment autonomously targets the insertion site, and performs the insertion without operator intervention. To aid in the design of a suitable device, the procedure is divided along the functional steps to examine, position, and insert. To be of use in a field environment, all the functional steps take place within a single, portable device, one that can be easily stored and attached to a patient. Although an external electrical power supply is acceptable, no external mechanical power will enter the system, and the device produces its own leverage during insertion. It is expected that the medic will perform any external connections to the catheter after the device has completed the operation, such as attaching the connections used for delivering the resuscitation fluids or other medication.

These and further features and advantages of the present invention will become apparent from the following detailed description, wherein reference is made to the figures in the accompanying drawings.

### BRIEF DESCRIPTION OF THE DRAWINGS

FIG. 1 illustrates the sonosite ultrasound transducer.

FIG. 2 illustrates the arc rotation manipulator concept.

FIG. 3 illustrates the spherical joint manipulator concept.

FIG. 4 illustrates the concentric multi-link manipulator concept.

FIG. 5 illustrates the CMS joint diagram.

FIG. 6 illustrates the diagram of the implement stack and clamping jaws.

FIG. 7 illustrates a first insertion mechanism.

FIG. 8 illustrates the first view of the second insertion mechanism.

FIG. 9 illustrates the second view of the second insertion mechanism.

FIG. 10 illustrates the third view of the second insertion mechanism.

FIG. 11 illustrates the initialization step configuration.

FIG. 12 illustrates the needle insertion stage.

FIG. 13 illustrates the dilator and needle retraction stage.

FIG. 14 illustrates the manipulator mechanism joint and linkage definitions.

FIG. 15 illustrates the plot of the effect of L₁/L₂ on joint loads.

FIG. 16 illustrates the linear drive length of travel diagram.

FIG. 17 illustrates the ADAMS model of the catheter insertion mechanism.

FIG. 18 illustrates the skin and tissue insertion model fitted with empirical data.

FIG. 19 illustrates the vein model parameters.

FIG. 20 illustrates the target insertion vein parameters.

FIG. 21 illustrates the plot of the gravity effects on the α joint.

FIG. 22 illustrates the plot of the gravity effects on the β joint.

FIG. 23 illustrates the plot of the maximum loads on the manipulator joints.

FIG. 24 illustrates the plot of the actuation torques for Simulation 1.

FIG. 25 illustrates the plot of the orientation errors for Simulation 1.

FIG. 26 illustrates the plot of the actuation torque for Simulation 2.

FIG. 27 illustrates the plot of the orientation errors for Simulation 2.

FIG. 28 illustrates the manipulator module prototype.

FIG. 29 illustrates the needle insertion force measurement setup.

FIG. 30 illustrates the experimental skin-vein model.

FIG. 31 illustrates the force data from needle insertions into the skin/tissue/vein model.

FIG. 32 illustrates the force data from needle insertions into the skin/tissue model.

FIG. 33 illustrates the force data from implement insertions into the skin/tissue model.

FIG. 34 illustrates the line detection algorithm frame.

FIG. 35 illustrates the manipulator orientation input-output performance.

FIG. 36 illustrates the imaging data obtained.

FIGS. 37-42 are each drawings of components for the Manipulator Module.

### DETAILED DESCRIPTION OF PREFERRED EMBODIMENTS

A catheter is a tube that can be inserted into a body cavity, duct, or vessel. Catheters thereby allow drainage, injection of fluids, or access by surgical instruments. The process of inserting a catheter is catheterization. Catheters may be thin, flexible tubes (soft) or in some cases larger and more solid (hard).

Catheter tube insertions allow, intra alia, IV access is as required for anesthesia care, laboring patients, trauma patients, hospital inpatients, and patient care requiring any of, but not limited to, the following therapies: emergency administration of medications, rapid infusion of fluids, especially blood products in critically ill patients, fluid resuscitation, elective administration of intravenous antibiotics, chemotherapeutic agents, other treatments and the administration of diagnostic substances, such as intravenous imaging or contrast agents.

Catheter or tube insertions typically involve the manual insertion of a hollow inducer needle through of which the catheter is manually inserted until the distal portion lies within the lumen of the vessel. The inducer needle is then carefully withdrawn and the catheter remains with one end in the vessel and the other outside the patient's body.

Alternatively catheter insertion may involve the manual insertion of a hollow inducer needle through which a guide wire is manually inserted until the distal portion of the guide wire lies within the lumen of the vessel. The introducer needle, which has the guide wire running through its length, is then carefully removed manually from the patient by pulling the needle out and over the guide wire, such that the distal end of the wire remains inside the lumen of vessel. The catheter is then manually slid over the proximal end of the guide wire, and the catheter is manually advanced along the wire into the vessel. Thus inserted, the catheter will have one end in the vein and the other end outside of the body. The guide wire is now removed by carefully pulling the wire out through the center of the catheter without disturbing the catheter.

The invention may be understood by splitting it into three subsystems, determined by the functional steps mentioned earlier. The first subsystem, referred to as the Imaging Module, is devoted to the diagnosis of the condition and the identification of the insertion region. In one embodiment this involves a modular ultrasound system, with a transducer linked to a mobile docking station, and an external laser scanner. The second subsystem, the Insertion Mechanism Module, performs a controlled insertion of the catheter. The last subsystem, the Manipulator Module, orients the Insertion Mechanism about the insertion region. The Manipulator Module may be referred to as the Positioning Module. Although the Imaging Module is largely self-contained, the division between the Manipulator Module and the Insertion Module may become blurred. For example, in the case of a robotic arm manipulating a needle, positioning and insertion are accomplished by the same mechanism. However, a preferred embodiment relies on a modular approach to the problem, and as such, all three subsystems can be treated as independent subsystems that are ultimately integrated into a fully-functional catheter insertion device. Each of these modules preferably interfaces with a central control system, which includes a computer.

Given the desired conditions previously mentioned, this embodiment solves or addresses the following concerns:

1. Mechanism can safely insert a catheter into the femoral vein using a modified, innovative version of the Seldinger technique.

2. Mechanism allows for a fully-automatic operation once the device has been placed in the desired insertion position.

3. Mechanism provides reliable and repeatable results.

The following procedures may be followed in the simulation and design of a fully-automated Catheter Insertion Mechanism that inserts a flexible catheter into the femoral vein.

The following paragraphs describe the particular anatomical and procedural aspects that define and drive the design of the Catheter Insertion Mechanism. Because the femoral vein is a likely target for insertion of a catheter, the local anatomy of the femoral vein is discussed, with particular interest on the geometric constraints it imposes on the design. This discussion is followed by a description of the current methods used to perform vein catheterizations, highlighting the typical complications medical practitioners encounter in practice. After this, the discussion focuses on the additional set of constraints added to the design process as a direct result of the battlefield environment in which the mechanism is expected to operate.

Human anatomy tends to vary significantly, depending on several factors ranging from age and gender, to physical fitness and genetic traits, and the femoral vein geometry is no exception. Thus, to mitigate the effects of anatomical variations, medical practitioners have identified one ideal site for femoral vein catheterizations. Located at the so-called femoral triangle, a recessed area in the medial aspect of the thigh just below the inguinal ligament (this ligament is easily identifiable as the crease formed at the groin), this region is often considered the optimal catheter insertion site for the femoral vein because in this region the vein is not covered by a significant amount of muscular tissue, leaving the vein conveniently exposed for catheterization. Even though the method has demonstrated to be quite effective throughout the years, geometric variations of the femoral vein depth, diameter, and overall body location usually lead to complications during femoral vein catheterizations. In 2000, Hughes et al. published the mean and range variation of femoral vein depth and diameter, as well as their variation at different distances away from the inguinal ligament in the inferior direction (the direction towards the feet), in a study that consisted of 50 patients (30 male, 20 female). The results are summarized in Table 1.

Aside from publishing femoral vein geometric data, Hughes et al. also mentioned that in their findings, the femoral vein was observed to “hide” behind the femoral artery at a distance of only 4 cm below the inguinal ligament, as opposed to the 10 cm distance commonly mentioned in standard anatomical texts.

TABLE 1
Femoral Vein Anatomy in Reference to the Inguinal Ligament

| | Vein Depth (cm)* | Vein Diameter (cm)* |
|---|---|---|
| At Ligament | 2.3 (0.5) [0.8-3.6] | 1.2 (0.3) [0.6-2.0] |
| 2 cm below Ligament | 2.0 (0.5) [1.0-3.1] | 1.1 (0.2) [0.6-1.7] |
| 4 cm below Ligament | 2.2 (0.5) [1.2-3.8] | 1.0 (0.3) [0.4-1.8] |

*Values are presented in the format: Mean (Standard Deviation) [Range]

Additionally, a separate study conducted by Seyahi et al. demonstrated that femoral vein depths increase as the Body Mass Index (BMI) of the patient increases. The data presents large geometric variations, and as such, it poses significant constraints to the Catheter Insertion Mechanism design. However, these variations in geometry can be accounted for with the addition of a visual feedback system (Imaging Module) that may monitor the actual location and size of the vein in real-time and thus help guide the catheter insertion process under uncertain conditions. This premise relaxes the functional requirements of the Manipulator and Insertion Modules, but it still requires the Manipulator Module to orient the Insertion Module in space and for the Insertion Module to insert the implements to reach vein targets located at a wide range of depths.

In common practice, medics typically place femoral vein catheters “blindly” using a landmark-based technique and tactile feedback to locate the vein. The surgeon begins by locating the groin crease marked by the inguinal ligament and begins to search for pulsations which mark the location of the femoral artery. Once the artery is properly located, the medic begins to insert a hollow needle with a syringe at about 1.5 to 2 cm to the medial side of the artery at an angle of approximately 20° to 45° with respect to the skin plane and in the superior direction (towards the torso), which commonly indicates the location of the femoral vein. The insertion depth, as defined in medical practice handbooks, is usually about 2 to 4 cm. The medic then checks for proper vein penetration by checking the hemostatic pressure of the blood owing in through the needle. Once the needle is properly inserted, the procedure followed is the Seldinger technique. In a controlled hospital environment, the whole procedure typically takes 2-3 minutes. Typical needle sizes used vary, but frequently 18G (1.27 mm±0.025 mm O.D., 0.838 mm±0.038 mm I.D.) or 20G (0.9081 mm±0.0064 mm O.D., 0.603 mm±0.019 mm I.D.) needles are used. Catheter sizes also vary greatly depending on the specific procedure to be performed, and typically range from 5Fr (1.67 mm O.D.) all the way to 30Fr (10.0 mm O.D.). It would be too large for a substantial portion of the population. However, given the restrictions posed by the nominal diameter of the femoral vein as presented by Hughes et al., a suitable catheter size is chosen to be 19Fr (6.3 mm O.D.) in order to be safely inserted into a femoral vein diameter that suits the majority of the population.

The uncertainty in the anatomy of the human body usually causes complications for the medic performing the catheterization. One of the most common complications is the accidental puncturing of the femoral artery, which may result in haematoma or false aneurysm. Additionally, if the catheter is inserted to an insufficient depth or placed incorrectly, extravasation of the infused solution into the surrounding tissue can occur. Sometimes the medic might also insert the needle too deep into the vein, penetrating the two vein walls completely (a condition commonly referred to as backwalling the vein), causing serious complications. For the above reasons, ultrasound imaging is usually utilized as an effective tool to verify the location of the vein and reduce the incidence of the complications arising from accidental, repeated, and incorrect insertions.

In addition to the possible complications that arise during the catheterization of the femoral vein, the target operating environment also poses an additional layer of issues that require attention. In austere far forward battle scenarios, portability and ruggedness are essential design specifications. Therefore, size constraints, as well as material considerations should be optimized to fulfill these requirements. Furthermore, the lack of available medics trained to perform vascular access procedures requires the design to be fully-automatic, or at least to be easily deployed by a medically-unskilled operator. Without a surgeon in the loop, an innovative method of locating the insertion site, inserting the required implements, and verifying a successful catheterization is required. In recent years, ultrasound has been used to provide reliable real-time visual feedback to guide surgical procedures. Therefore, it is postulated that the enhanced precision of a robotic system, coupled with the capability of using insertion force and visual feedback to guide the insertions, provides enough reliability to perform an automated catheterization of the femoral vein, even in the battlefield.

### Imaging Module

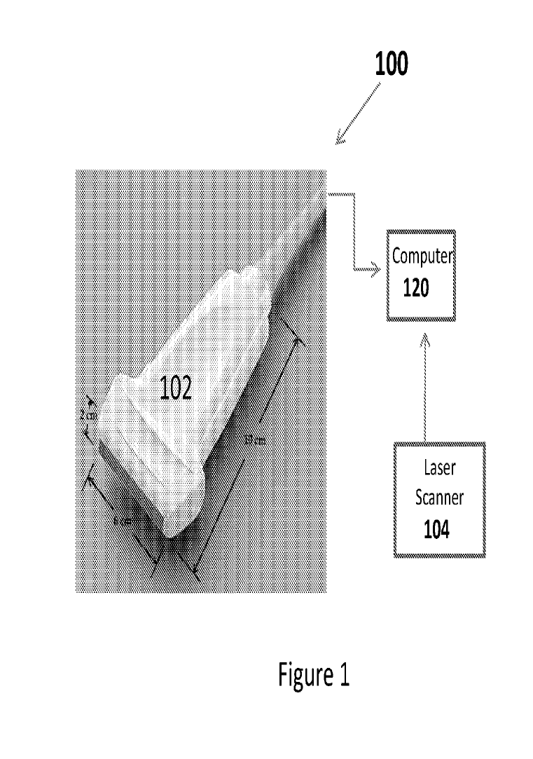

A suitable Imaging Module 100, as shown in FIG. 1, mainly consists of a linear-array ultrasound (US) probe or scanner 102, to be placed anywhere in the area surrounding the target insertion point. The US probe considered for this development was a Sonosite Titan, Model L38, which will geometrically span an area rectangle of 6 cm by 2 cm along the surface of the pelvic region. The body of the transducer, ergonomically shaped for hand-held use as shown in FIG. 1, extends over 10 cm high, above the skin surface. A preferred imaging module includes both an ultrasound probe 102 for viewing internal body parts and a laser scanner 104 for imaging relevant locations on the outside of the patient. The ultrasound scanner provides information about the location of the vessels within the patient, and this information is used to control fine movements of the Manipulator Module and to guide movements of the Insertion Module. A preferred system may use a 2-D planar ultrasonic probe 102. Ancillary equipment may include a laser scanning system that identifies the general area where insertion will occur. This scanner may provide the initial partial body map for moving the system into position, preferably using an X-Y frame or an arm. A suitable scanner is the NextEngine 3D scanner. For the purposes of this system, a more robust system with faster scanning capabilities may be desired to efficiently demarcate the zone of interest for direct intervention. Figures for both the ultrasonic device 102 and the laser scanner 104 may be input to a computer 120, which may then output signals to the Manipulator Module. The Catheter Insertion Mechanism design does not significantly obstruct the area surrounding the insertion point to provide the Imaging Module with enough freedom to guide the insertions.

The Imaging Module directs and verifies movement of the Insertion Module to align with the femoral artery or vein. This module also provides guidance and feedback throughout the insertion procedure by identifying and tracking the components as they move towards the target vessel. Ultrasound imaging is well suited for this application due to its small size, low power requirement, robust construction, lack of harmful radiation and ability to detect areas of flow, which is a particularly valuable tool when attempting to identify blood vessels.

Advancements in ultrasound technology, both in Doppler flow analysis and 2 dimensional phased arrays, have brought the modality to the point where 3 dimensional anatomical data may be received and processed on a machine the size of a laptop. Examples include the GE Vivid i and Sonosite Titan. 2D and 3D ultrasound systems are already used by clinicians to identify blood vessels and guide needle insertion by hand. Thus, there is little problem finding hardware fit for the task at hand. The challenge lies in the automation of these tasks with high reliability.

Ultrasound is gaining acceptance as an effective means of accurate cannulation. Blood vessel detection may be performed using an ultrasound system by attempting to locate the morphology of the vessel, namely an object with a nearly circular cross section. Pulse color Doppler flow measurement is another tool that may be used for vessel detection. By measuring changes in frequency, areas of flow may be mapped on top of conventional ultrasound images. Vessel lumina light up brightly when measured for flow. In addition, the direction and amount of flow may be observed using the Doppler method. This allows for the distinction of vessel type, i.e., vein or artery. Veins have a low flow value with a dampened flow waveform, while arteries have higher flowrates with higher amplitudes. Distinguishing a vein from an artery is important, particularly when dealing with the femoral area since the vein and artery are located very close to each other. Image processing techniques for tracking a needle have been studied by a number of groups, with many using 2D ultrasound or fluoroscopy as the imaging modality. The presence of speckle in ultrasound systems demands a robust image processing technique.

Portable clinical devices intended for direct human interaction are not designed for rapid image transfer to another computer for image processing beyond the capabilities of the unit itself, nor are many equipped to receive control signals from another computer. Tactile buttons and dials may be provided for adjusting the parameters and modes of the system. A workaround using clinical devices, or the use of an OEM unit, may be used to provide adequate control and data throughput to the image processing system. Data transfer protocols such as TCP/IP over gigabit Ethernet may provide the bandwidth for 3D volumes.

Having a clearer picture of the problem at hand allows one to define the most important constraints and requirements which will ultimately drive the design of the mechanism. Following this premise, the problem has been condensed into a function-oriented, qualitative and, to some degree, quantitative breakdown of the overall design constraints and requirements. The results are summarized in Table 2. These design constraints and requirements are further addressed in the subsequent concept development and selection stages of the design of the modules that constitute the Catheter Manipulator and the Catheter Insertion Modules.

TABLE 2
Constraints and Requirements for Catheter Insertion Mechanism

| Category | Constraint/Requirement |
|---|---|
| Geometry | Insert 18G needles and up to 19Fr catheters into femoral vein |
| | Angle of insertion, ψ, must satisfy: 20° < = ψ < = 45°. |
| | Insertion site must be open for Imaging Module to operate. |
| Operational | Design must complete catheterization in less than 15 sec. |
| | Design must inherently prevent backwalling. |
| Portability | Design volume must be no more than 25 cm³, |
| | when not in use. |
| | Design must weight no more than 4.5 Kg. |
| Flexibility | Design must orient the insertion arbitrarily in space. |
| | Design must insert implements to a wide range of depths |

### Manipulator Module

The design of the Manipulator Module was treated as an independent subsystem, and was designed, for most purposes, independently of the Insertion Module. A modular design makes the fabrication of the Manipulator Module simpler. Thus, in order to design the manipulator, one may first define the specific functional design constraints and requirements that drive the focus of the design. Alternative concepts may be developed to suit these functional needs, and ultimately, the optimal candidate may be selected in accordance with predefined performance measures. The sections that follow outline this process in detail.

The functional requirements and constraints of the Catheter Manipulator

### Module are:

Securely supports the weight and the quasi-static loads applied to the Insertion Module during the catheterization process.
Orients the needle and implements arbitrarily in space, using the minimal required Degrees-of-Freedom (DOFs) about the point of insertion.
Allows for needle and implements to remain steadily fixed in space during insertion.
Mechanism does not obstruct the area near the insertion region.
Design weight is under 2.5 Kg.

Given the functional requirements established in the previous section, three possible concepts were developed and evaluated against each other to ultimately yield the best concept possible. The three concepts are further discussed below.

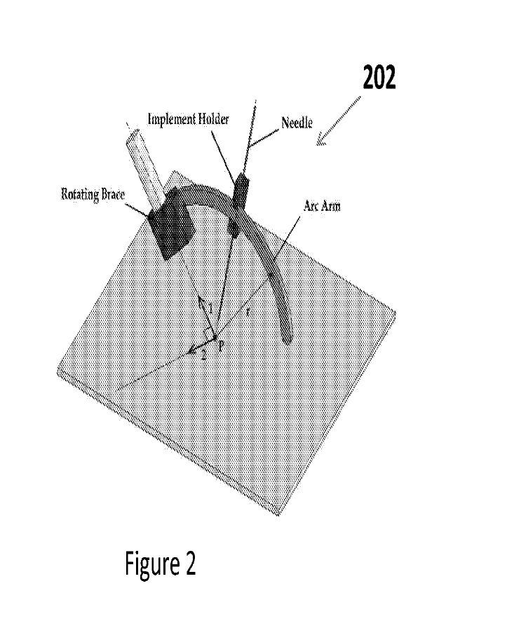

Concept 1 is denoted as the Arc Rotation Manipulator (ARM) Concept 202. As shown in FIG. 2, the design consists of a brace that rotates the rest of the mechanism about axis 1 at the insertion point P, and an implement holder representing the Catheter Insertion Module, which translates around the arc arm at a radius r.

The motion of the Insertion Module along the arc arm is designed in such a way as to create a rotation about axis 2 through point P. The two rotations about axes 1 and 2 are thus orthogonal and fixed about the distant point P, which provides the two required DOFs about the insertion region. The main advantage of this design is its simplicity to design and manufacture. Some drawbacks, however, are the possible inability of the design to hold an Insertion Module of considerable weight due to possible binding of moving parts that constitute the arc motion, and the large arc arm size required to provide the required angle of insertion.

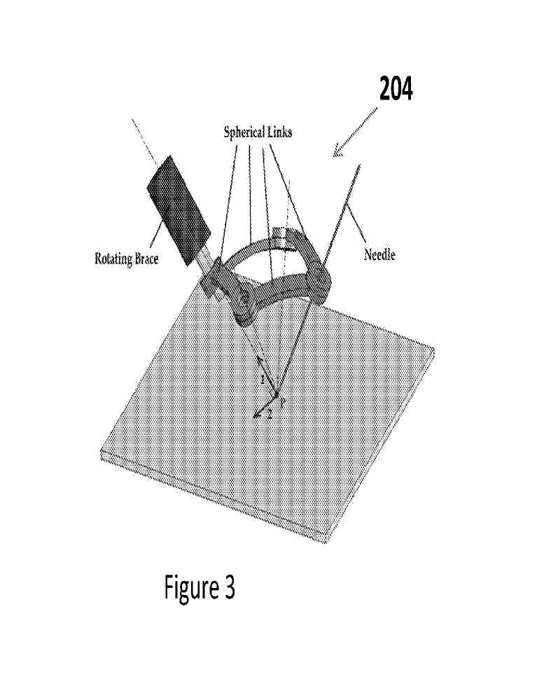

Concept 2 is denoted as the Spherical Linkage Manipulator (SLM) Concept 204. As shown in FIG. 3, this design consists of a brace that rotates the rest of the linkage mechanism about axis 1 at the insertion point P, and a set of circular arc-shaped links with equal radii and designed in such a way as to provide a rotation about axis 2 at Point P.

The main advantage of this design is the precision that may be achieved if the links are adequately manufactured. The main disadvantage is the fact that this design may not provide enough rigidity to hold the Insertion Module, particularly when the linkage is extended to large insertion angles. One way to increase the rigidity of this mechanism would be to increase the mass of the joints and linkages, which is highly undesirable. Furthermore, the precision of the design and manufacturing stages should be relatively high since any interference or miscalculation may render the linkage difficult or even impossible to move.

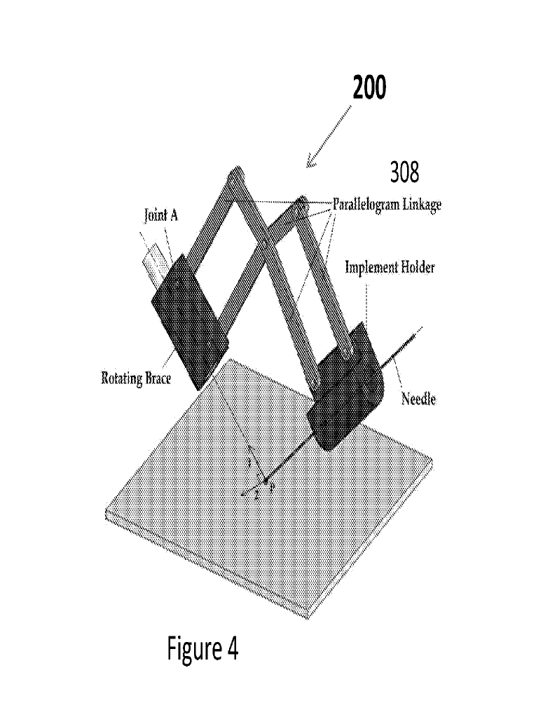

Concept 3 is denoted as the Concentric Multi-link Spherical (CMS) Manipulator 306 and consists of a dual-parallelogram linkage mechanism 308 that provides the two required orthogonal DOFs much like the spherical joint about the insertion point P, as shown in FIG. 4. The rotation about axis 1 is created by the motion of the rotating brace, which moves the whole linkage about the point of insertion, P. The second DOF is created by the design of the linkage and by the actuation of Joint A, which creates a rotation about axis 2 at point P. The main advantages of this design are its enhanced rigidity as compared to both Concepts 1 and 2, as well as its design flexibility due to the fact that the link lengths may be adjusted to optimize the loads on the linkage joints. Its principal drawback is its complexity of design and manufacture when compared to the other presented concepts.

The incorporation of a dual parallelogram linkage mechanism in the present device creates a Remote Center of Motion (RCM) about the insertion point. This parallel linkage concept provides the advantage that it enables the actuation of the degree-of-freedom that corresponds to the insertion angle (the angle of the needle axis with respect to the skin surface) from the relatively-fixed base of the linkage. Thus, the present device incorporates three degrees-of-freedom about the insertion point, which orients the needle at any desired position within the workspace of the mechanism itself. Because each patient's vein axis is unlikely to lie in the same orientation, the addition of a dual parallelogram linkage mechanism in the present device allows maximal flexibility in orienting the needle to a greater variety of unknown vein axis orientations. Absent this degree of flexibility, among other things, one would have to ensure that the patient's vein axis lies within the insertion plane before beginning the insertion procedure to avoid missing or passing through the target vessel.

The concepts presented in the previous section were evaluated according to the following metrics (ranked in order of significance):
1. Insertion Precision
2. Efficient Mobility
3. Geometric Size
4. Fabrication Complexity
5. Innovative Concept

Insertion precision refers to the anticipated ability of the manipulator concept to consistently position the catheter insertion mechanism at the desired orientation, without significant inherent and foreseeable errors resulting from the loads imposed by the weight of the Insertion Module or the insertion forces. Efficient mobility includes the ease of mobility of the mechanism (smoothness of motion, without significant obstructions and restrictions), as well as the size of the working space derived from the specific kinematic motions of each design concept. Geometric size refers to the approximate effective volume each concept is expected to occupy. Fabrication complexity refers to the estimated machining and assembly time required for each concept, as well as the complexity inherent in the design itself. Finally, innovation covers the capability of the concept to be adapted to future applications.

Each of the concepts was then evaluated using SolidWorks 3D Models to evaluate Insertion Precision, Efficient Mobility, Geometric Size, and Fabrication Complexity. A ‘+’, ‘0’, or ‘−’ mark was given to each concept for each metric, where a ‘+’ denotes a favorable point, a ‘0’ denotes a neutral mark and no points, and a ‘−’ denotes a negative point. The points were then added and the concept with the highest number of points was selected as the viable design. The selection process is summarized in Table 3. As it can be seen from Table 3, Concept 3, referred to as the CMS Manipulator 306, is the most-suitable choice for the Manipulator Module 200. This design concept is further discussed and validated through the simulation discussed below.

TABLE 3
Manipulator Module Design Selection Metrics

| Metric | Concept 1 | Concept 2 | Concept 3 |
|---|---|---|---|
| Insertion Precision | − | + | + |
| Efficient Mobility | − | 0 | + |
| Geometric Size | − | + | 0 |
| Fabrication Complexity | + | 0 | − |
| Innovative Concept | 0 | − | + |
| Total | −2 | 1 | 2 |

Once the final concept is selected, several design-specific considerations and computations should be addressed in order to validate the feasibility of the design analytically. Thus, in this section, the basic kinematics of the mechanism are discussed to verify if the motion of the manipulator satisfies the specified motion requirements.

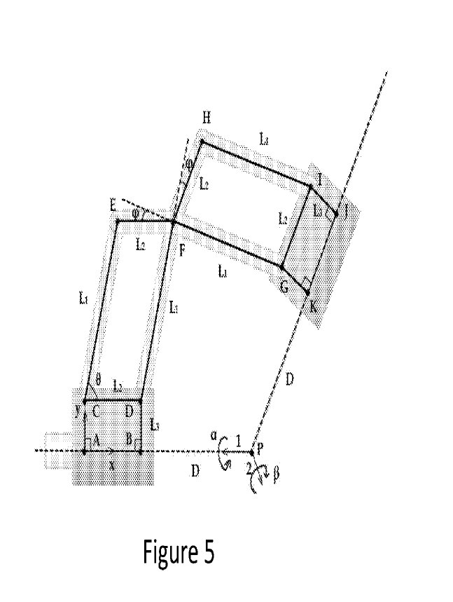

In order to better understand and verify the motion of the CMS Manipulator, a kinematic analysis was performed to demonstrate that the multi-link joint indeed provides the desired DOFs required for this application. The first rotation (defined as the a rotation) is trivial in its analysis since it is evident that it provides a Remote Center of Motion (RCM) rotation about axis 1 through the insertion point, P. Refer to FIG. 5 for the notation used. The second RCM rotation (denoted as the β rotation), however, is not as evident. Thus, a kinematic analysis was performed as follows.

Given the link length parameters L₁, L₂, and L₃, the following are the corresponding linkage dimensions:

$$\begin{matrix}\overline{CE} = \overline{DF} = \overline{FG} = \overline{HI} = {L}_{1} & Eq . 1 \\ \overline{AB} = \overline{CD} = \overline{EF} = \overline{FH} = \overline{GI} = \overline{JK} = {L}_{2} & Eq . 2 \\ \overline{AC} = \overline{BD} = \overline{GK} = \overline{IJ} = {L}_{3} & Eq . 3 \\ \overline{BP} = \overline{KP} = D & Eq . 4 \\ φ = arctan \frac{{L}_{3}}{D} & Eq . 5 \\ {L}_{1} = \frac{{L}_{3}}{sin φ} & Eq . 6\end{matrix}$$

Furthermore, it is important to emphasize the following geometric constraints:

A̅B̅∥ C̅D̅∥ E̅F̅ Eq. 7

C̅E̅∥ D̅F̅Eq. 8

F̅H̅∥ G̅I̅∥ J̅K̅Eq. 9

F̅G̅∥ H̅I̅Eq. 10

The kinematic analysis consists of determining the location of the intersection point of the extension lines formed by segments AB and JK, with respect to a fixed rectangular reference frame centered at Point A, as shown in FIG. 5. This is the point labeled P on the diagram. If this point of intersection remains fixed for any given input orientation angle, θ, and if the length of segments BP and KP remain equal and constant, then Point P is the Remote Center of Motion (RCM) of the manipulator, thus providing the second DOF required for arbitrary orientation. The location of Point K is defined as a sum of the linkage vectors in rectangular coordinates with respect to the fixed reference frame centered at Point A, as follows:

$$\begin{matrix}K = {R}_{1} + {R}_{2} + {R}_{3} + {R}_{4} + {R}_{5} & Eq . 11 \\ {R}_{1} = {L}_{2} \hat{i} & Eq . 12 \\ {R}_{2} = {L}_{3} \hat{j} & Eq . 13 \\ {R}_{3} = {L}_{1} cos θ \hat{i} + {L}_{1} sin θ \hat{j} & Eq . 14 \\ {R}_{4} = {L}_{1} cos φ \hat{ i} + {L}_{1} sin φ \hat{j} & Eq . 15 \\ {R}_{5} = {L}_{3} cos ( θ - φ - \frac{π}{2} ) \hat{i} + {L}_{3} sin ( θ - φ - \frac{π}{2} ) \hat{j} & Eq . 16\end{matrix}$$

By further simplification and substituting Equations 4 and 5 into the above expression, the following equation is obtained:

$$K = \frac{( D + {L}_{2} ) {L}_{1} + ( {L}_{1}^{2} - {L}_{3}^{2} ) cos θ + {L}_{3} D sin θ}{{L}_{1}} \hat{i} + \frac{( {L}_{1}^{2} - {L}_{3}^{2} ) sin θ - {L}_{3} D cos θ}{{L}_{1}} \hat{j} \qquad Eq . 17$$

Since the extended line created by Points A and B is coincident with the x-axis of the fixed reference frame, and the location of P may also be defined at some point along the extended line created by Points J and K, the location of P may be expressed as:

P=K−λ(cos(θ−φ)i+sin(θ−φ)\hat{j} Eq. 18

In this equation, λ is the parameter that defines how far along JK the point P is located. To find out where this line intersects the x-axis, one may find the value of A for which the y-component of the position of Point P is zero:

$$\frac{( {L}_{1}^{2} - {L}_{3}^{2} ) sin θ - {L}_{3} D cos θ}{{L}_{1}} - λ sin ( θ - φ ) = 0 \qquad Eq . 19$$

Solving for λ yields the following:

λ=D Eq. 20

Furthermore, by substituting this value into Equation 18, the following result is obtained:

P=(L₂+D)\hat{i} Eq. 21

Therefore, because both the location of P and the value of λ are both independent of θ, one may conclude that the point P is indeed the Remote Center of Motion for the manipulator, thus providing the required motion for arbitrary catheter insertions.

The Manipulator Module may include robotic elements for aligning and stabilizing the insertion and imaging modules with respect to the target area of the subject. Proper function of the imaging and insertion modules involves close contact with the skin of the subject in the groin area. Additionally, the imaging module is able to move itself to align its field of view with the axis of the blood vessel to be cannulated, such that the vessel may be seen longitudinally. Thus, the manipulator module has the ability to cover a rather large area while also having the capability to perform minute adjustments to get the other modules into position. The motorized Concentric Multi-Link Spherical (CMS) manipulator system pivots the imaging and insertion modules as a unit relative to the subject. Movement of the CMS manipulation module may be planned and executed by the central control system, using a global coordinate system that integrates visual feedback from the laser scanner (gross movement across subject) and the ultrasound scanner (fine movement across subject). Additional feedback mechanisms may improve the accuracy of movement by the manipulator module. Once the imaging and insertion modules are in the correct position, the manipulator module may remain in position throughout the insertion procedure, and sustains the loads associated with the procedure.

### Catheter Insertion Module

The design effort also included the Catheter Insertion Module. In this section, two candidate design concepts are presented and one is selected as the Insertion Module design based on predefined performance measures. The needle preferably uses an echogenic surface treatment or coating to enhance its visibility under ultrasonics.

The first step in the concept development stage is to recognize the desired functional structure of the design by analyzing the current steps taken by the medic to perform a successful vascular access procedure using the Seldinger technique. These steps are then grouped and converted into the functional structure sequence of the Insertion Module, outlined below:
Grasp the needle and insert until vein is reached
Insert and hold needle in place
Insert dilator over needle until dilator is inside the vein
Hold dilator in place and retract the needle
Insert catheter over dilator while holding the dilator
Hold catheter in place and retract dilator
Remove mechanism leaving catheter in place

The functional structure sequence generally follows the Seldinger technique, with two major exceptions. First, the functional sequence does not include the step in which the surgeon creates an incision into the patient to widen the insertion site. Instead, the Insertion Module will rely on the use of a tapered dilator to gradually open up the insertion site as it is introduced into the patient.

A dilator is a device used to stretch or enlarge an opening used to make the access hole larger. The use of a dilator allows the use of an inducer needle that is much smaller than the diameter of the final catheter. This is advantageous because the use of a smaller inducer needle causes much less tissue trauma should multiple insertion attempts be required. This is particularly important when trying to access arteries, because of the increased risk of hematomas and pseudoaneurysm. The possible issue of a smaller inducer needle being harder to visualize on ultrasound is overcome, among other ways, by providing an echogenic surface treatment to the inducer needle. The possible issue of the thinner inducer needle flexing as it penetrates tissue and deviating in the direction of the bevel is overcome with a closed-loop control using image extraction from the ultrasound probe. Furthermore, a larger access hole allows the insertion of a catheter tube of increased gauge that has a larger inner diameter, which augments the rate at which fluids can be infused. This ability may be particularly helpful in trauma patients. Furthermore, the use of the dilator to enhance the size of the opening allows the insertion of a catheter tube that has a more rounded less beveled tip, providing a greater margin of safety by decreasing the chance of passing the catheter through the distal wall of the vessel during insertion for example. The reduction of flow restriction which would accompany the broader tip would also reduce the likelihood of turbulence and thus, among other things reducing the induction of micro-thrombi.

The other major step deleted from the Seldinger technique is the use of the guidewire. Hand threading a wire through the inside of a needle is a quite complicated procedure to reproduce mechanically. Thus, instead of using a guidewire, a series of implements of increasingly larger diameters is radially stacked and inserted into the patient in a multi-step insertion procedure that relies on the precision of the insertion mechanism drive, without the need of adding additional DOFs, keeping the design simple and portable. This radially stacked group of implements is referred to as the implement stack. First, the needle is inserted into the patient, and while the needle is held in place, a tapered rigid dilator with an inner diameter slightly larger than the inner diameter of the needle is inserted over the needle shaft. Once the dilator is introduced to the desired position inside the femoral vein, the needle may then be retracted through the dilator. The catheter tube, with a inner diameter slightly larger than the outer diameter of the dilator, is inserted into its final position inside the vein while the dilator is held in place. Finally, once the catheter is safely inside the vein, the dilator is retracted, leaving the catheter inside the vein.

The Insertion Module is a complex mechanism, and two exemplary mechanisms were fabricated.

The first mechanism was created at UT Austin and was tested as a device for the insertion of chest tubes into the human thoracic cavity, and the second mechanism is a design concept that was tested as a device intended to obtain vascular access of the femoral vein. The two concepts are discussed below.

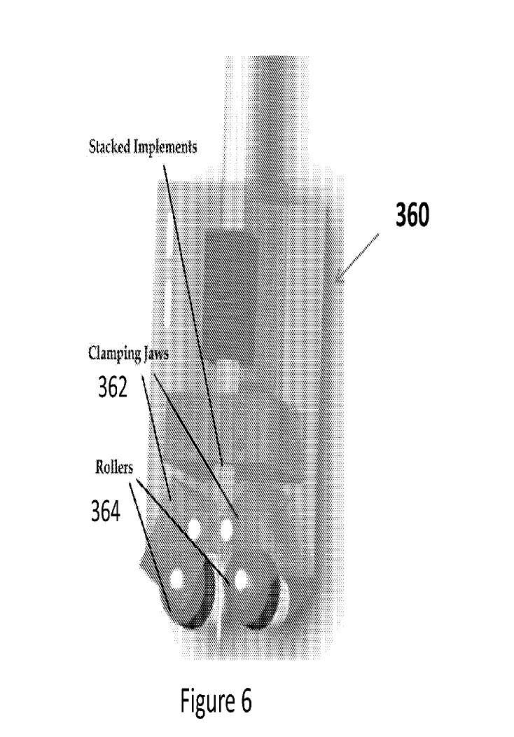

The first insertion mechanism 360 relies in the implement stacking procedure as shown in FIG. 6. In this mechanism, each implement is inserted into the body with the aid of a set of gripping jaws 362 and a roller drive 364. The implement is first gripped by a set of independently actuated jaws that clamp down on it until a desired grip force is reached. Once the implement is in full grip, the rollers are actuated to drive the implement into the body, relying on the friction between the rollers and the implement. If at any point during the insertion the implement stops moving and the rollers begin to slip, the rollers stop rotating and the jaws clamp tighter and tighter on the implement until the rollers are able to move the implement again.

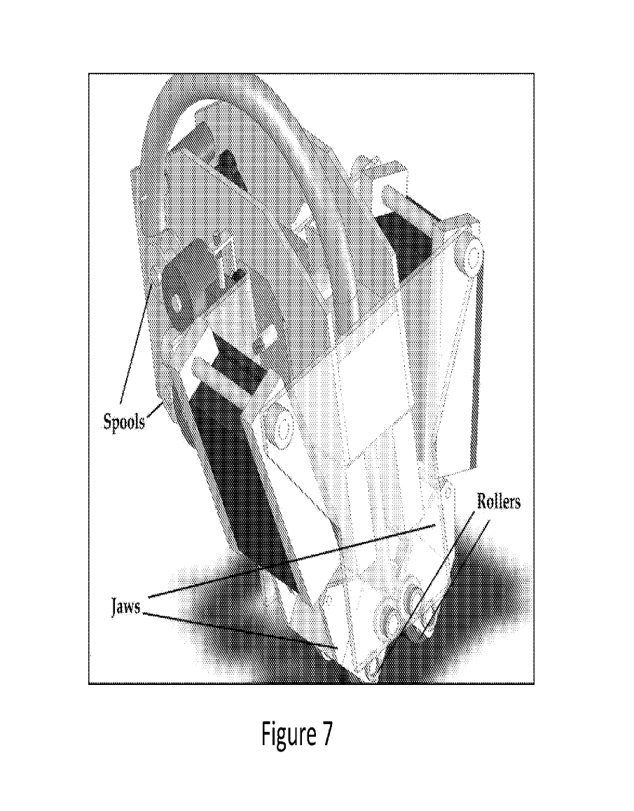

In order to hold the implements in place and to retract them when needed, this design also uses a set of spools with wires attached to the top end of each implement, as shown in FIG. 7. Each spool is driven by a motor and coupled to a clutch. The motor is used to retract each implement by reeling the wire attached to that implement. The clutch is actuated in order to prevent the spool from being reeled out during the insertion, whenever this is desired. The main advantage of this design is the innovative use of the spool system to hold each implement in place and to reel them in as necessary. However, one of the main disadvantages of this concept is its large size. Furthermore, another particular problem with using frictional rollers to drive the implements into the patient is the fact that insertion forces cannot be measured directly in a simple and reliable manner with this configuration. This aspect is significant to this application because direct force feedback may greatly aid in the guidance of the catheter insertion. Furthermore, during operation, if the mechanism detects slippage between the rollers and the implements too frequently, the jaws tend to clamp down so hard on the implements that they deform and are unable to be inserted any further.

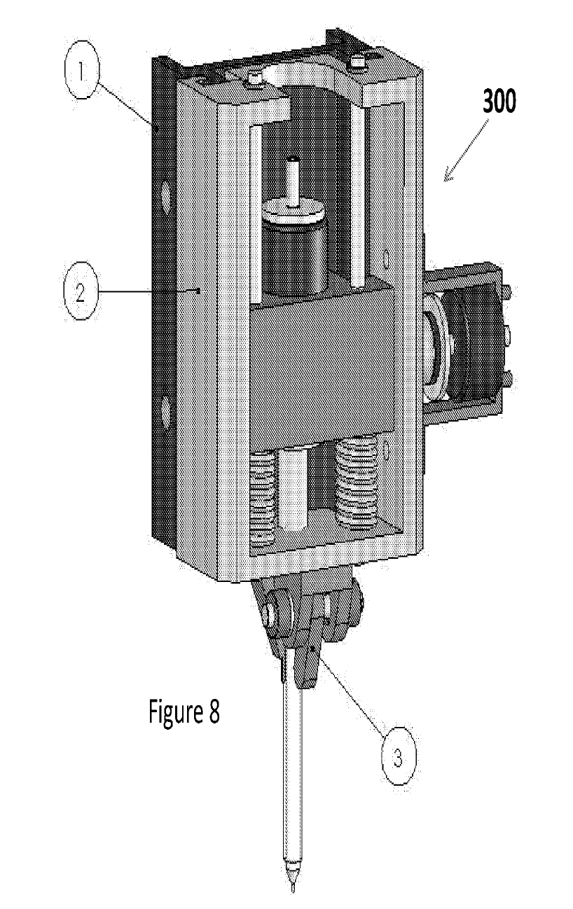

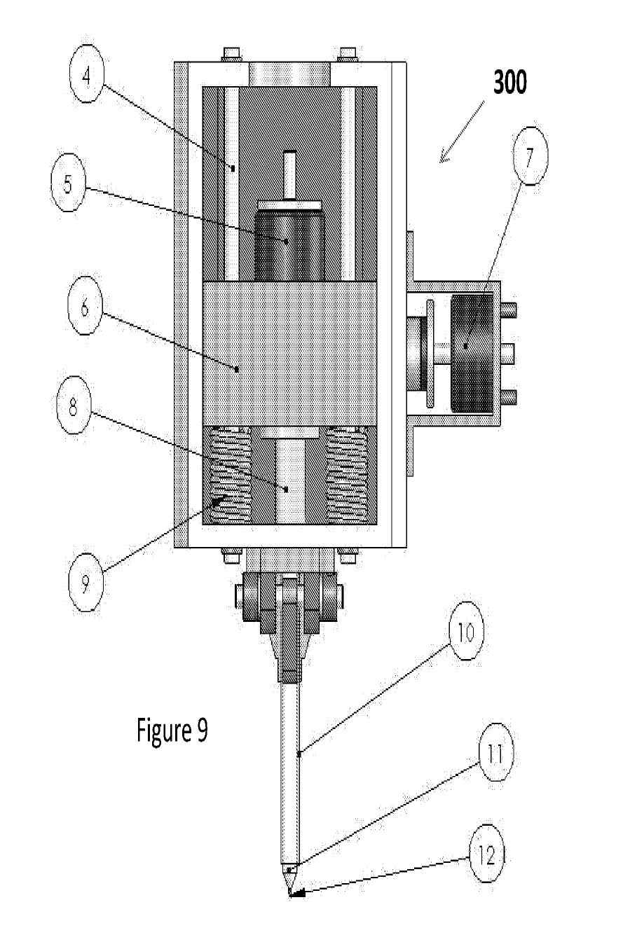

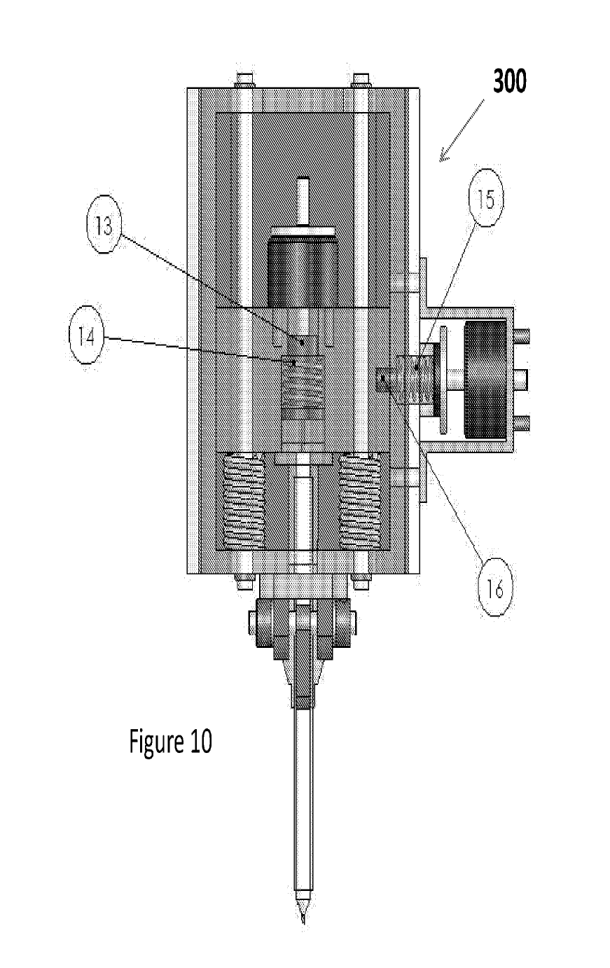

The second insertion embodiment 300 also relies on the stacking procedure discussed above to avoid using the guidewire. In this design, each implement is inserted into the body with a linear drive, as opposed to the roller design. Furthermore, the insertion of the needle and dilator is combined in one step to simplify the insertion procedure and circumvent the need to retract each implement using wires or any other form of full retraction. The embodiment is shown in different views in FIGS. 8, 9, and 10.

Following is a brief description of the functions of each labeled part in FIGS. 8-10 and provides the naming convention used later to refer to each part during the discussion of the catheter insertion procedure:

1. End-effector Bracket: This bracket is attached to the Manipulator Module. This bracket interlocks with the Catheter Drive Housing (2) to provide the linear motion that drives the Needle (12) into the vein via a rack and pinion assembly.

2. Catheter Drive Housing: This part houses all the parts of the mechanism.

3. Implement Gripper Arm: This part provides additional support to the implements (Needle, Dilator, and Catheter Tube). The gripper also consists of a clamping arm that is used to hold the Catheter Tube (10) in place.

4. Guide Rails: The rails provide the smooth translation of the Implement Retraction Base (6) during Dilator (11) retraction.

5. Needle Retraction Solenoid: This solenoid, when actuated, pushes the Needle (12) tip outside of the Dilator (11). When not energized, the Needle Retraction Spring (14) retracts the Needle (12) inside the Dilator (11).

6. Implement Retraction Base: This block houses the Needle (12) and the Dilator Holder (8).

7. Locking Pin Retraction Solenoid: This solenoid, when actuated, pulls the Locking Pin (16) out, which releases the Implement Retraction Base (6), retracting both the Needle (12) and the Dilator (11).

8. Dilator Holder: This part attaches to the Implement Retraction Base (6) and holds the Dilator (11).

9. Dilator Retraction Springs: When the Locking Pin (16) is released, these compression springs extend upwards, retracting the Needle (12) and the Dilator (11), which are attached to the Implement Retraction Base (6).

10. Catheter Tube

11. Dilator

12. Needle

13. Needle Holder: Attached to the Needle (12), this part receives the load of the Needle Retraction Solenoid (5) during insertion and maintains the Needle (12) retracted inside the Dilator (11) when the Needle Retraction Solenoid (5) is not energized.

14. Needle Retraction Spring: This extension spring holds the Needle (12) retracted when the Needle Retraction Solenoid (5) is off.

15. Locking Pin Return Spring: This extension spring keeps the Locking Pin (16) inside the Implement Retraction Base (6) to keep it from being released.

16. Locking Pin: Holds the Implement Retraction Base (6) in place.

During the Catheter Insertion Procedure description that follows, references to the list above are denoted by referencing the corresponding part number inside the parentheses.

The first step, denoted as Stage 0, is the initialization, or off-state of the mechanism. In this state, the active elements are operated as follows:

1. Linear Drive is off. Therefore, the catheter drive housing (2) does not move relative to the end-effector bracket (1).

2. Needle Retraction Solenoid is off. Thus, the needle (12) tip is located inside the dilator (11) because when this solenoid is off, the needle retraction spring (14) holds the needle in this position.

3. Locking Pin Retraction Solenoid is off. Thus, the pin is inserted into the implement retraction base (6), holding it in place, and the dilator retraction springs (9) remain compressed.

4. Gripper arm is not engaged.

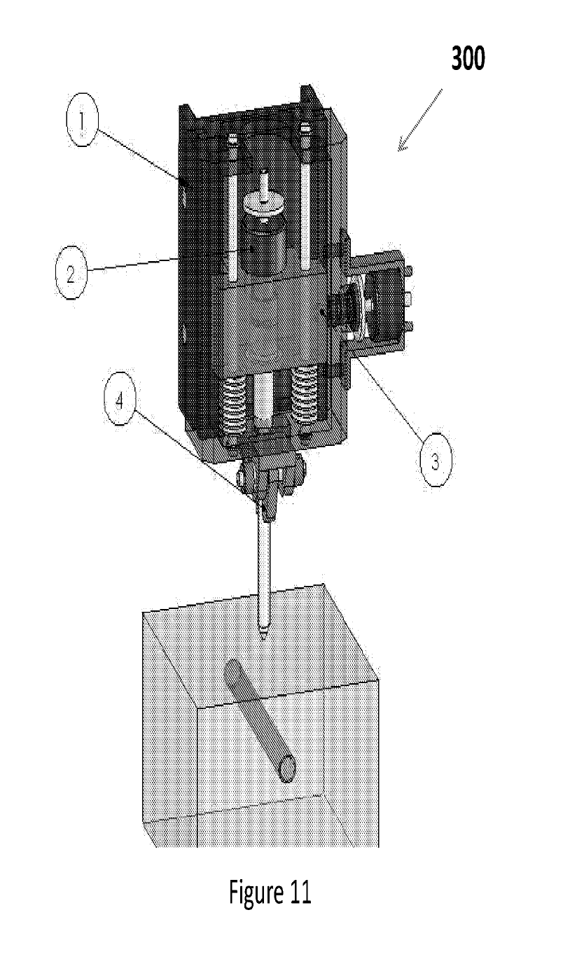

FIG. 11 illustrates the actual configuration of the parts during this stage (Note the numbers in the diagram correspond to the steps shown above).

Once the drive mechanism is positioned in the desired orientation, Stage 1, The Insertion Stage, begins. During this stage, the following steps are followed in order:

1. Gripper Arm (3) is engaged. This helps keep the implements fixed and helps avoid buckling or bending of the implements during insertion.

2. Needle Retraction Solenoid (5) is energized. Powering this solenoid pushes the needle holder (13) down, extending the needle retraction spring (14) and exposing the needle tip outside the dilator to allow for insertion.

3. Linear Drive is engaged. Once steps 1 and 2 are completed, the whole catheter drive housing (2) is then linearly driven into the patient.

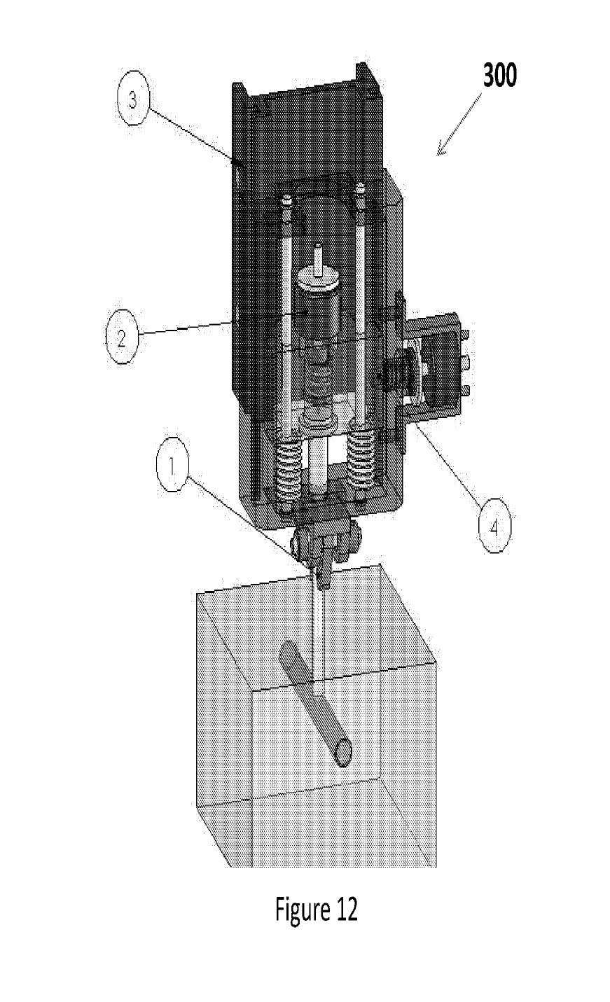

4. Locking Pin Retraction Solenoid (7) remains off. Step 3 in this sequence is performed until the vein has been punctured and the vein and dilator are inside. FIG. 12 shows the final configuration of the mechanism at the end of Stage 1.

Stage 2 constitutes the Back-Walling Prevention Stage. Once the needle and the dilator puncture through the vein, the needle is retracted inside the dilator to prevent it from puncturing the back wall of the vein and to be able to drive the dilator inside the vein further. This is done by turning the Needle Retraction Solenoid (5) off. This will allow the needle retraction spring (14) to contract and pull the needle tip inside the dilator. Once the needle tip is retracted, the linear drive is engaged until a suitable dilator insertion depth is detected and the catheter tube is inside the vein. Once the tube is located inside the vein, in Stage 3, the needle and the dilator may be retracted by following the steps below:

1. Leave the gripper arm (3) engaged to ensure that the catheter tube is held in place while the needle and dilator are retracted.

2. Leave the Needle Retraction Solenoid (5) off.

3. Leave the linear drive off.

4. Engage the Locking Pin Retraction Solenoid (7). This will retract the locking pin that holds the implement retraction base (6) in place and thus enable the dilator retraction springs (9) to push the base away from the needle insertion site, retracting both the needle and dilator, which are attached to it.

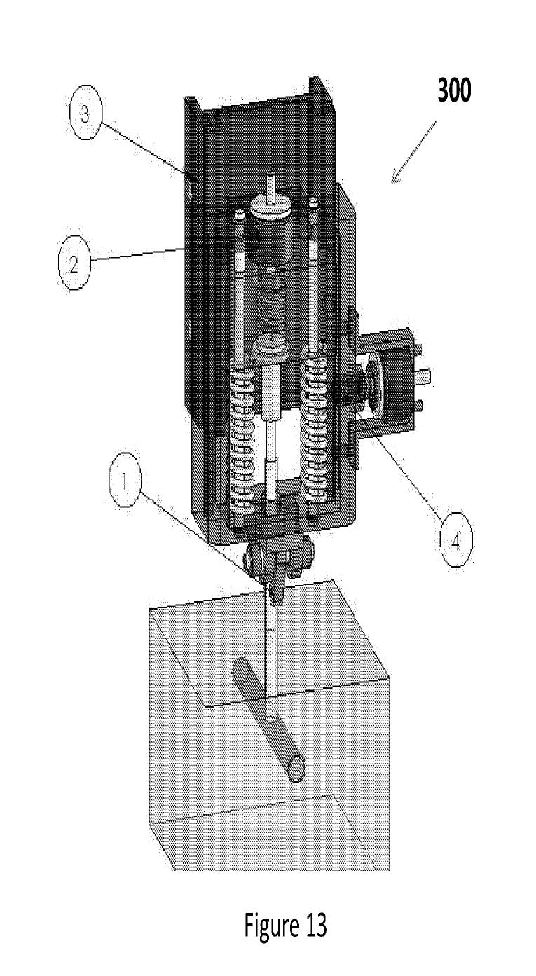

The tube itself does not retract with the other implements because it is not attached to any part of the retraction base assembly and it is held in place by the gripper arm (3). A diagram of how this step works is shown in FIG. 13.

Stage 4 concerns the steps taken once the tube is inside the vein. In this stage, further insertion depths may be reached by simply actuating the linear drive while holding the gripper arm (3) actuated. However, if the end of the linear drive travel is reached, further insertion depths may be achieved by releasing the gripper arm (3), then back driving the linear drive away from the insertion site a predefined distance, then actuating the gripper arm again, driving the tube again into the insertion site. These steps may be performed until the final tube position is reached. Finally, in Stage 5, the gripper arm is released and the whole mechanism removed, leaving the catheter tube in place.

The main advantage of this design over the alternative first insertion mechanism is its more simplified insertion approach, coupled with a smaller profile. One major disadvantage, however, is the increased expected insertion forces that may result from the simultaneous insertion of the implements into the patient.

Although each of the embodiments offered enough promise for further exploration in detail, time and resources warranted the use of metrics to choose the concept to pursue first. Listed in Table 4 are the five categories by which the concepts were judged. The first, Insertion Precision, estimates the ability of the mechanism to place the catheter in the target femoral vein with minimal error. Next, is Force Feedback, which ranks the capability of the design to include a reliable and simple force feedback measurement to help guide the insertion. The third category is Geometric Size, which involves the predicted overall volume occupied by the mechanism. Next is Fabrication Complexity, which deals with the overall prototype fabrication and design time. A simpler solution usually receives a better score. Lastly, Innovation, looks at the capability of adapting the design to future applications. The scoring system is the same as the one implemented in the selection of the Manipulator Module design. The results of the selection process are also presented in Table 4.

TABLE 4
Insertion Module Design Selection Metrics

| Metric | Concept 1 | Concept 2 |
|---|---|---|
| Insertion Precision | 0 | + |
| Force Feedback | 0 | + |
| Geometric Size | − | + |
| Fabrication Complexity | − | 0 |
| Innovative Concept | 0 | + |
| Total | −2 | 4 |

Using these performance metrics, the second Insertion Mechanism Concept (Concept 2) was selected. With this selection, validation through simulation of the Insertion Module is discussed in detail below.

The Insertion Module may use a series of implements to dilate a needle puncture to the diameter required to insert the final catheter using a multi-step insertion technique. A combination of solenoids, motors, springs and rotary encoders may be used to perform these steps. Several techniques for driving the implements through tissue have been explored, including a friction roller method, whereby two adjustable rollers simultaneously grip and drive insertion implements. The adjustable nature of the rollers allows them to grip various diameter implements. However, this technique has limitations and drawbacks, including slippage of the rollers that may occur under heavy puncture resistance by the tissue. Accounting for the lack of movement due to slippage may be difficult. A preferred method of insertion relies on a more direct application of force, through direct linear translation of the implements. The various implements may be stacked radially within each other, and may be sequentially driven down into tissue.

### Needle Insertion Simulation

This section includes validating the design, either analytically through theory and simulation or experimentally via prototype design. In order to minimize production costs and to optimize the design before engaging in construction, the design was verified via simulation using ADAMS™ Dynamic Simulation Software developed by MSC Software, Inc. First, a planar simulation was used to determine the optimal design link length parameters that will be used for the construction of the Manipulator Module. After the Manipulator geometry is fully defined, the geometric parameters involved in the simulation of the Insertion Module are also further discussed. Furthermore, the quasi-static simulation is further extended to the three-dimensional case by including the α RCM rotation discussed above. This extended model is used to determine the actuation torques for each of the two RCM rotations, α and β (created by actuator rotations, θ₁ and θ₂, respectively) required to hold the mechanism in place, referred to in this chapter as the gravity effects. Finally, the analysis is unified in a comprehensive model that performs a PID-controlled needle insertion into a simulated model of the pelvic region and the results are discussed in the context of the insight provided to the design of the Catheter Insertion Mechanism.

One of the main advantages of using analytical methods in design is the ability to make multiple design modifications and evaluate the improvements on the design.

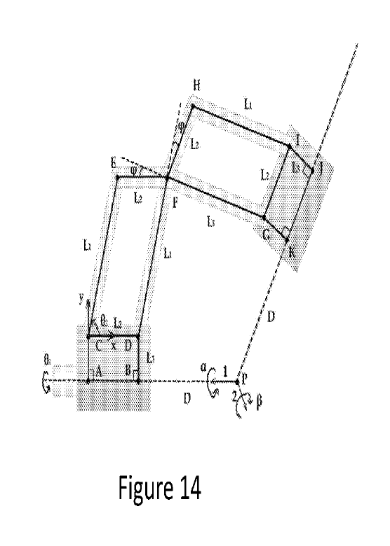

MSC.ADAMS™ provides the capability to run multiple sequential repetitive simulations that serve to analyze the effects of one or multiple parameters on a particular set of output metrics, such as torque and force measurements. Using this capability, several batch simulations were performed to determine the effect of each of the independent manipulator link lengths, L₂ and L₃ (Lᵢ is derived from L₃), on the static torque sensed at the β actuation joint located at Point C, as well as the forces sensed at each other joint in the mechanism. Refer to FIG. 14 for the joint definitions.

The ultimate goal of the Design of Experiments (DOE) simulation is to determine the optimal link lengths that minimize the joint loads required to actuate the linkage. To determine the geometry of the Insertion Module, a simpler approach is followed due to the fact that the most significant size limitations and requirements are directly correlated to the anatomical geometry of the insertion region, as well as to the geometry of the implements to be inserted, i.e. needle, dilator, and catheter.

In order to perform the DOE simulation of the manipulator, each joint location in the manipulator linkage was parameterized in ADAMS™. Using the naming convention demonstrated in FIG. 14, each joint location was defined using rectangular coordinates established with respect to the fixed reference frame located at point C. The simulation is planar and thus, the actuation angle defined as θ₁ in FIG. 14 is not included in the analysis and in order to simplify the notation, the θ actuation rotation angle, θ₂ is here referred to as simply θ. The x and y coordinates for each point Pᵢ centered at Joint i are presented in Table 5.

TABLE 5
Location of Manipulator Joints in Rectangular Coordinates Point
Coordinates X Y

| Point | X | Y |
|---|---|---|
| P_{C} | 0 | 0 |
| P_{D} | L₂ | 0 |
| P_{E} | L₁ cos θ | L₁ sin θ |
| P_{F} | L₂ + P_{D}_{_{X}} | P_{E}_{_{Y}} |
| P_{H} | P_{F}_{_{X}} + L₂ cos(θ − φ) | P_{F}_{_{Y}}+ L₁ sin(θ − φ) |
| P_{I} | P_{H}_{_{X}} + L₁ cos φ | P_{H}_{_{Y}} + L₁ sin φ |
| P_{G} | P_{F}_{_{X}} + L₁ cos φ | P_{F}_{_{Y}} − L₁ sin φ |
| P_{J} | P_{I}_{_{X}} + L₃ cos(θ − φ − π) | P_{I}_{_{Y}} + L₃ cos(θ − φ − π) |
| P_{K} | P_{G}_{_{X}} + L₃ cos(θ − φ − π) | P_{G}_{_{Y}} + L₃ sin(θ − φ − π) |

Once the coordinates are defined, design variables are created for the two independent link variable parameters, L₂ and L₃. The simulation is created such that when each of these two values is modified, the links and joints are updated automatically to retain the same RCM kinematic motion. Furthermore, a measure is created to track the magnitude of the forces at Joints C-K, for a fixed angle θ=45°. In order for the simulation to provide feasible practical results, the geometric and size constraints of the mechanism should be added to the simulation. First, the overall mechanism is required to fit inside a volume of 25 cm³, which puts upper bounds on the size of the linkages. Additionally, in order for the mechanism to have enough joint and actuator clearance, some of the links should also be constrained to be within certain predefined lower bounds. L₁, for instance, should be constrained to be no larger than the maximum allowed length of 25 cm. Therefore, L₁ should be lower than 120 mm to accommodate the length of the fully extended mechanism, which is close to 2L₁ at full extension. Furthermore, L₃ should provide enough clearance space for Joints C, D, I, and G, which is estimated at a minimum of 20 mm, while still maintaining a relative small mechanism size, for which 30 mm is considered a suitable upper bound. The distance from the end-effector edge of the mechanism to the insertion site, denoted as D, is also constrained to be at least 110 mm to allow enough clearance for the major component of the Imaging Module, the ultrasound probe, which, as discussed above, spans 10 cm in height. The geometric constraints are therefore defined as:

L₁≦120 mm Eq. 22

20 mm≦L₃≦30 mm Eq. 23

D≧110 mm Eq. 24

Furthermore, L₂ defines the length of the base of the mechanism, and even though it has no explicit geometric constraints, there is a kinetic constraint identified in the analysis, which states that there are limits on the ratio of link lengths L₁ and L₂ that should be used in order to mitigate the non-uniformity of the reaction forces at different joints. The ratio is defined as:

$$\frac{1}{2} \leq \frac{{L}_{1}}{{L}_{2}} \leq 2 \qquad Eq . 25$$

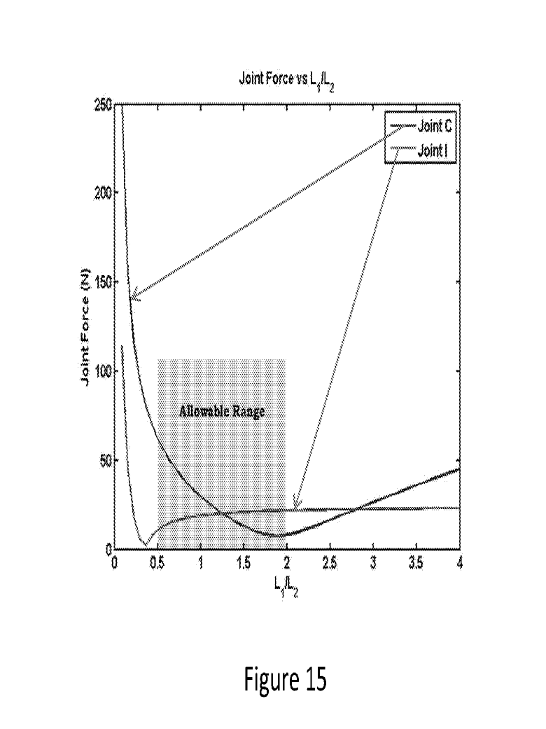

In order to verify this conclusion, a preliminary simulation was performed in order to analyze the effects of the ratio in question on the magnitude of the joint force on two representative joints, Joint C and Joint I. The joints were selected because they reflect each other about the line of symmetry of the mechanism. FIG. 15 shows the results from the simulation. As it may be seen from the plot, the allowable region identified by Faraz and Payandeh marks an important tradeoff between L₁ and L₂. Within this region, as the ratio increases, the magnitude of the forces on Joint C decreases but the magnitude of the forces on Joint I increases, and viceversa. The pattern of Joint I may also be measured on Joints H and G, and the behavior of Joint C may also be observed in Joints E and D, which makes intuitive sense due to the symmetry of the linkage. Therefore, this constraint was also added to the simulation.

The optimization simulation was constrained with the expressions previously discussed. The optimization problem is thus defined as:

min_{L}_{₂}_{,L}_{₃}∥F∥₂ Eq. 26

F=|fᵢ|∀i=C,D, . . . , H,I Eq. 27

where fᵢ is the two-dimensional force vector sensed at Joint i, and the ∥•∥₂ notation represents the I²-norm of the given argument.

This objective function was selected to minimize the overall joint forces. The resulting parameter values from the simulation are summarized in Table 6 (The values have been rounded off).

TABLE 6
Manipulator Linkage Parameters

| Parameter | Dimension |
|---|---|
| L₁ | 120 mm |
| L₂ | 60 mm |
| L₃ | 30 mm |
| D | 116 mm |
| φ | 14.5° |

The geometric parameters of the Insertion Module are constrained by the anatomy of the insertion region, and thus the size synthesis of this module may be almost completely derived directly from the values established in Table 2. Each particular parameter definition is discussed in detail in the subsequent sections.

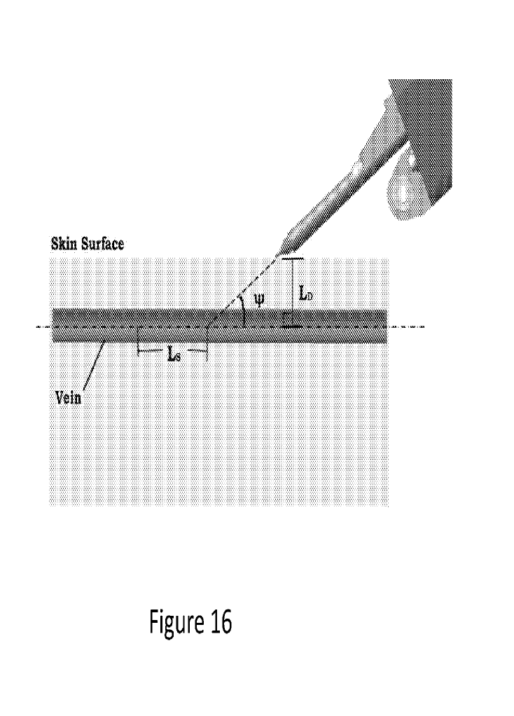

The Linear Drive of the Insertion Module as discussed above may be composed of a rack and pinion assembly that creates the relative linear motion between the End-effector Bracket and the Insertion Mechanism Housing. The length of travel of the drive should be sufficient to insert the needle/dilator/catheter stack down into the target vein, an average vertical distance, L_{D}, of 2.3 cm at the inguinal ligament, according to Table 2.1. In computing this depth, however, the insertion angle should also be accounted for. Refer to FIG. 16. In computing the minimal required travel length, consider the worst-case scenario in which ψ=20°. Furthermore, once the implement stack is inserted to the desired depth, the catheter tube should be inserted further into the vein to prevent it from slipping out once the mechanism is released. Surgeons typically suggest that the catheter should be inserted a safety distance, L_{S}, of approximately 4 cm. Therefore, the Linear Drive should have an overall travel length, L_{T}, equal to:

$${L}_{T} = \frac{{L}_{D}}{sin ψ} + {L}_{S} \approx 10.75 cm \qquad Eq . 28$$

The dimensions of the implements are defined by the geometry of the insertion site. First, the catheter tube outer diameter should be equivalent to a 19Fr catheter, which is approximately 6.3 mm, and its inner diameter should be equivalent to the outer diameter of the dilator tube to allow the stacking of the implements, which forces the catheter tube's inner diameter to be approximately 4.75 mm. Furthermore, the catheter tube overall length should be at least the distance L_{T} defined in the previous section, plus an additional 5 cm so that the catheter tube protrudes from the skin surface to allow a medic to administer drugs and fluids, making an overall distance of about 15.75 cm. The dilator tube, should have an outer diameter equivalent to 4.75 mm and an inner diameter equivalent to the outer diameter of the 18G needle, which is approximately 1.27 mm. Additionally, the dilator length should be equivalent to the catheter tube length, plus a stacking offset of approximately 3 mm, which makes it about 16.05 cm long. Finally, the needle outer diameter and bevel geometry should be equivalent to an 18G needle with a 30° bevel tip and its length should be equivalent to the length of the dilator, plus the additional stacking offset of 3 mm, which makes it approximately 16.35 mm long.

Once each of module geometries have been defined, the next step is to use those models in a position-control simulation. The goal of the simulation is twofold. First, the simulation should provide an accurate insight into the two actuation torques required to orient the Insertion Module, as well as aid in the selection of the mechanical components required by the design, such as return spring constants, solenoid holding torques, and bearings and linkage materials. Secondly, the simulation should provide a reliable testbed for experimenting with different control schemes in order to find the best control strategy. Thus, the simulation is composed of three main parts:

1. The Catheter Insertion Mechanism Dynamics

2. The Skin and Vein Model

3. The Control Algorithm Model

Each part is discussed in detail in the subsequent sections.

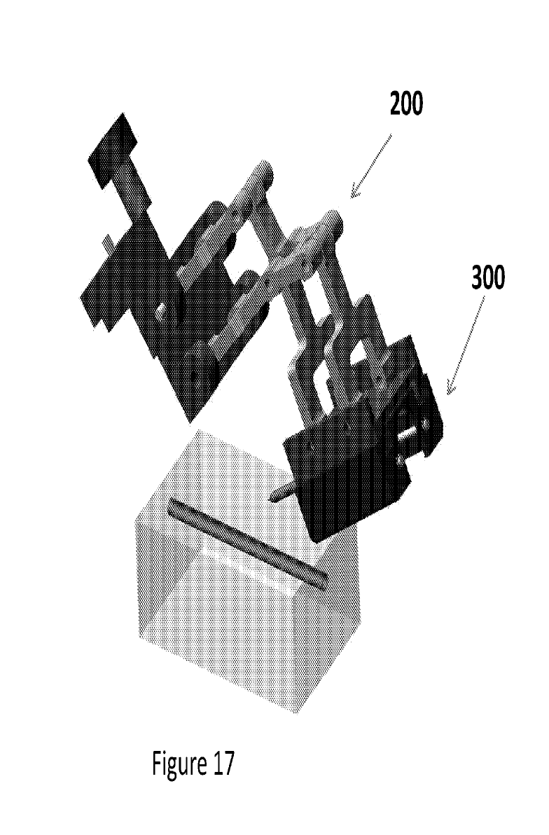

Two of the key features of MSC.ADAMS™ are the capability of importing design parts as parasolid files that accurately maintain the geometric and physical properties of the design and its capability of assembling each part into a dynamically accurate model by adding physical constraints, such as rotational and translational joints, and body contact constraints. Taking advantage of these features, the model of the Catheter Insertion Mechanism, as shown in FIG. 17, models the kinematics of the design. The kinematic constraints feature rotational joints between each of the link joints of the Manipulator module, a rotational joint to model the a rotation of the linkage, a translational joint to model the motion of the linear drive of the Insertion Module relative to the Manipulator Module, and an additional translational joint to model the relative motion between the catheter drive housing and the implement retraction base along the guide rails during dilator and needle extraction. Refer to FIGS. 8-12 for a list of the part names of the Insertion Module discussed here. The kinetic constraints of the design features applied torques to the α and β rotational joints to use for position control, as well as the addition of two spring components to simulate the dilator retraction springs and one more spring component added to simulate the needle retraction spring. Furthermore, the needle retraction spring solenoid is modeled by adding an appropriate holding force on the needle holder to push the needle out of the dilator when energized and removing the force when the solenoid is not energized. The same logic is applied to model the locking pin retraction solenoid, except that the holding force is initially applied when the solenoid is in its off state and removed when the solenoid is turned on. Finally, two contact constraints are modeled, the first between the catheter tube and the dilator holder to simulate the pushing of the holder against the catheter tube, and the second between the catheter tube and the implement gripper arm to simulate the gripper arm holding the tube in place during the insertion process.

In order to fully simulate the insertion procedure, the interaction between the insertion implements and the skin and vein tissues should be accurately modeled to reflect the results from experimental insertion data. Several models have been presented in the literature to model these interactions but few, if any, provide a definitive modeling solution. However, there are some common denominators between different models. The studies conducted by Simone and Okamura and Maurel, for instance, divide the insertion process into a pre-puncture and a post-puncture phase. In pre-puncture, the axial force on the needle tip increases steadily due to the nonlinear elasticity of the surface tissue and a sharp drop in the amount of force identifies the puncture event. In Simone and Okamura, this initial increase in force is modeled as a nonlinear spring of the second order polynomial form:

f(d)=a_{θ}+a₁d+a₂d² Eq. 29

Alternatively, Maurel models the same phenomena with a nonlinear spring of the exponential form:

f(d)=(F_{θ}+b)e^{a(d-d}^{_{θ}}⁾+b Eq. 30

During post-puncture, Simone proposes that the amount of force is variable due to friction, cutting and collision with interior structures. As such, the post-puncture model is of the form:

f(d)=f_{friction}+f_{cutting} Eq. 31

where friction consists of a modified Karnopp friction model and f_{cutting} is a constant empirical value. Maurel, on the other hand, models the post-puncture phase as an exponential function of insertion depth similar to its pre-puncture phase.

The skin and vein insertion model used in the simulation expands on the principles proposed by Simone and Maurel using the contact constraints capabilities of MSC.ADAMS™. The model is divided into the pre-puncture and post-puncture stages. In the pre-puncture phase, the approach outlined by Eq. 29 is implemented in the simulation to find the best fit to experimental data. In the post-puncture phase, the force-displacement model is enhanced from the model proposed by the addition of a new term to account for the resistive force caused by the compression of the skin as the needle displaces the tissue and opens up the wound. This force is termed as the clamping force, and is defined as acting in the direction normal to the wall of the needle shaft. The complete post-puncture model is therefore defined as:

$$\begin{matrix}f ( x , y ) = {f}_{friction} ( x ) + {f}_{cutting} + {f}_{clamping} ( y ) & Eq . 32 \\ {f}_{friction} = \{ \begin{matrix}{C}_{n} sgn ( \dot{x} ) + {B}_{n} \dot{x} & \dot{x} \leq - Δ v \\ max ( {D}_{n} , {F}_{a} ) & - Δ v < \dot{x} < 0 \\ min ( {D}_{p} , {F}_{a} ) & 0 < \dot{x} < Δ v \\ {C}_{p} sgn ( \dot{x} ) + {B}_{p} \dot{x} & \dot{x} \geq Δ v\end{matrix} & Eq . 33\end{matrix}$$

where Cₙ and Cₚ are the negative and positive values of dynamic friction, Bₙ and Bₚ are the negative and positive damping coefficients, and Dₙ and Dₚ are the negative and positive values of static friction, respectively. x is the relative velocity between the needle and tissue, Δv is the value below which the velocity, x, is considered to be zero, and Fₐ is the sum of non-frictional forces applied to the system.

f_{clamping}(y)=a₀+a₁y+a₂y² Eq. 34

where y is defined as the skin deflection in the direction normal to the shaft of the needle and the aᵢ coefficients are fit parameters.

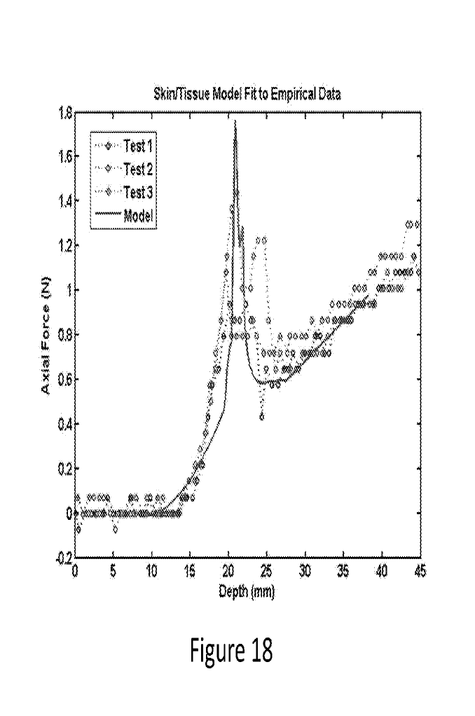

Once the model is established, empirical data defines the model parameters. This task is not trivial because although several studies documented in the literature have characterized the nature of the insertion forces into a myriad of biological organs, there are still no definitive insertion force measurement studies for procedures involving skin/fat/vein tissues. In the absence of any experimental data, an experimental setup was arranged to measure the insertion loads for a needle insertion into a simulated skin and vein tissue. However, as further explained below, a suitable experimental model to simulate the vein was not found and thus only the skin and tissue insertion data was used in the simulation. The experimental procedure is covered in detail below and only the resulting data is used here to fit the model. The fitted model and the empirical data are plotted in FIG. 18. The simulated model is denoted by the thick, dark blue line and the other lines are data obtained from the insertion experiments.

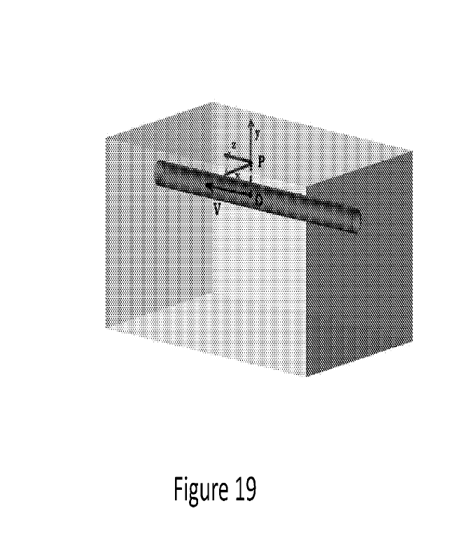

With the models of the Insertion Mechanism and the skin/tissue completed, the next step in the simulation is to model the control algorithm used to position the Insertion Module at the desired orientation based on the location feedback provided by the Imaging Module. Since an ultrasound probe was unavailable for this particular study, it is assumed that the Imaging Module is able to provide the location of the centroid of the vein cross-section, O, as well as the absolute position of a point, P, located on the skin surface directly above the vein centroid and the directional unit vector of the vein axis, V, all with respect to a global reference frame. Refer to FIG. 19. Thus, assuming the aforementioned parameters are fully defined, an algorithm was developed to determine the desired reference angles corresponding to the α and β rotations of the linkage mechanism required to orient the Insertion Module in a configuration that is adequate to insert the needle. The algorithm does the following:

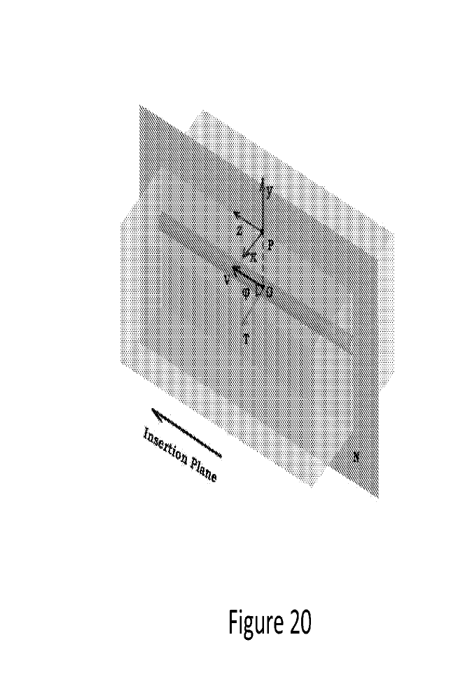

1. Uses the given vein orientation unit vector, V, to calculate a target orientation unit vector, T. Refer to FIG. 20.

2. Using T, the inverse position kinematics problem is solved to retrieve a and R.

3. α and β are used to compute the reference kinematic inputs to the controller, θ₁ and θ₂, which correspond to α and β, respectively.

As shown in FIG. 20, the target vein vector, T forms a plane, denoted as Plane N, with V that is perpendicular to the global x-z plane. This plane also bisects the vein along its axis and thus ensures that the target path defined by T intersects the vein. This puts constraints on vector T. Given that the coordinates (with respect to the global frame) of the vein directional unit vector, V, are defined as:

V=[V_{X}V_{Y}V_{Z}] Eq. 35

The target insertion directional unit vector, T, is defined as follows:

T=[V_{X}T_{Y}V_{Z}] Eq. 36

Thus, the vector T has only one unknown, the y-coordinate, T_{y}. This unknown may be computed using the fact that it is desirable to insert the needle at an angle, denoted as Φ, relative to the vein directional vector V. Mathematically, there are two possible solutions for which T and V form an angle Φ in Plane N. Using the definition of the dot product, the y-component of the first solution, T₁, may be computed by numerically solving the following equation for T_{1y}:

$$\begin{matrix}\frac{{T}_{1} \cdot V}{‖ {T}_{1} ‖ ‖ V ‖} - cos φ = 0 & Eq . 37 \\ {T}_{1} = \frac{{T}_{1}}{‖ {T}_{1} ‖} & Eq . 38\end{matrix}$$

The second solution of T may be visualized as the reflection of the T₁ vector obtained from Eq. 54 about V on Plane N. The expressions below formally define this relationship:

$$\begin{matrix}{T}_{2} = - {T}_{1} + 2 V ( {T}_{1} \cdot V ) & Eq . 39 \\ {T}_{2} = \frac{{T}_{2}}{‖ {T}_{2} ‖} & Eq . 40\end{matrix}$$

Out of the two possible solutions, the physically feasible solution is the one for which the following condition is true:

T_{Y}<V_{Y} Eq. 41

Therefore, the solution that satisfies the condition is the actual target orientation directional unit vector, T. Once the target orientation is defined, the next step is to solve the inverse kinematics problem to find the two orthogonal rotations that orient the mechanism to align with vector T. The Manipulator Module has two rotational DOFs, a about the z-axis of the global frame and β about the x-axis of the rotated frame. Given these two motions, the rotation matrix, R is:

$$ℝ = [ \begin{matrix}1 & 0 & 0 \\ 0 & cos β & sin β \\ 0 & - sin β & cos β\end{matrix} ] [ \begin{matrix}cos α & sin α & 0 \\ - sin α & cos α & 0 \\ 0 & 0 & 1\end{matrix} ] \\ ℝ = [ \begin{matrix}cos α & sin α & 0 \\ - sin α cos β & cos β cos α & sin β \\ sin β sin α & - sin β cos α & cos β\end{matrix} ] \qquad Eq . 42$$

In matrix R, each column represents the rotated locations of the rotated axes with respect to the global frame. In particular, column 2 represents the location of the rotated y-axis, which is conveniently oriented along the direction defined by vector T. Therefore, the following holds true:

$$[ \begin{matrix}sin α \\ cos β cos α \\ - sin β cos α\end{matrix} ] = [ \begin{matrix}{T}_{X} \\ {T}_{Y} \\ {T}_{Z}\end{matrix} ] \qquad Eq . 43$$

Using the relationships above, the following equations were derived for the Euler rotations:

$$\begin{matrix}α = arctan 2 ( {T}_{X} , - \sqrt{{T}_{Y}^{2} + {T}_{Z}^{2}} ) & Eq . 44 \\ β = arctan 2 ( \frac{- {T}_{Z}}{cos α} , \frac{{T}_{Y}}{cos α} ) & Eq . 45\end{matrix}$$

The Euler rotations, α and β are the desired input reference orientation angles used in the position control loop.

In order to assure a smooth motion from the initial orientation to the desired final orientation defined by θ₁ and θ₂ and to avoid actuator saturation, a robotic application typically requires the definition of via points that trace the prescribed trajectory. Several methods exist to determine these intermediate points, but for this simulation, the polynomial interpolation technique is preferred for its simplicity in derivation and implementation. First, the via points are uniformly spaced in time between the desired time elapsed between the initial orientation time at tᵢ, and the final orientation time, t_{f}, here defined as T:

T=t_{f}−tᵢ Eq. 46

Using a cubic interpolating polynomial, the following equations define the via points for the position and velocity trajectories:

$$\begin{matrix}{θ}_{{D}_{i}} ( t ) = {a}_{i} + ( t - {t}_{i} ) {b}_{i} + {( t - {t}_{i } )}^{2} {c}_{i} + {( t - {t}_{i} )}^{3} {d}_{i} & Eq . 47 \\ {\dot{θ}}_{{D}_{i}} ( t ) = {b}_{i} + 2 ( t - {t}_{i} ) {c}_{i} + 3 {( t - {t}_{i } )}^{2} {d}_{i} \\ where , & Eq . 48 \\ {a}_{i} = {θ}_{i} ( {t}_{0} ) & Eq . 49 \\ {b}_{i} = {\dot{θ}}_{i} ( {t}_{0} ) & Eq . 50 \\ {c}_{i} = \frac{3 [ {θ}_{i} ( {t}_{f} ) - {θ}_{i} ( {t}_{0} ) ] - T [ 2 {\dot{θ}}_{i} ( {t}_{0} ) + {\dot{θ}}_{i} ( {t}_{f} ) ]}{{T}^{2}} & Eq . 51 \\ {d}_{i} = \frac{2 [ {θ}_{i} ( {t}_{0} ) - {θ}_{i} ( {t}_{f} ) ] - T [ {\dot{θ}}_{i} ( {t}_{0} ) + {\dot{θ}}_{i} ( {t}_{f} ) ]}{{T}^{3}} & Eq . 52\end{matrix}$$

The equations above may be applied to each control angle, θ₁ and θ₂ independently. The trajectories are implemented into MSC.ADAMS™ using splines constructed from the given parameters automatically at the start of the simulation based on the input reference orientation angles, α and β.

Once the required joint positions are known, then the manipulator should move in this direction to properly orient the needle for insertion. To move the mechanism to the desired reference orientation, a modified Proportional-Integral-Derivative (PID) control scheme is implemented. The reason why a simple classical PID controller is not implemented in this simulation is because the PID controller tends to have a slow response time and in some cases tends to cause instability, particularly for long motion ranges and in cases where the gravitational effects on the mechanism are not negligible. To describe the nature of the controller, the manipulator dynamics is first briefly discussed. Classical robot rigid body dynamics texts, such as and usually represent the general rigid body dynamics of a robotic mechanism with ideal actuators and no joint friction in the form:

τ(θ)=M(θ)\ddot{θ}+V(θ,\dot{θ})+G(θ) Eq. 53

where the M(θ) term is the manipulator inertia matrix, V(θ;θ) represents the centrifugal and Coriolis terms, and G(θ) stands for the gravity terms. A commonly studied classical robotic control strategy for robotic manipulators is the so called Proportional-Derivative (PD) computed-torque control method. This control algorithm is a model-based approach that makes use of a control law of the form:

τ(θ)=M(θ)[\ddot{θ}_{D}+K_{D}\dot{ε}+K_{P}ε]+V(θ,\dot{θ})+G(θ) Eq. 54

where,

ε=θ_{D}−θ Eq. 55

\dot{ε}=\dot{θ}_{D}−\hat{θ} Eq. 56

In Eq. 55, the term θD refers to the desired orientation angle.

The computed-torque PD algorithm described above relies on accurate knowledge of the manipulator dynamic model, particularly the M(θ), V(θ,θ), and G(θ) terms. Ideally, if the dynamic model accurately captures the physical dynamics of the manipulator system, then position control may be readily achieved. In practice, however, accurately modeling the dynamics of complex robotic systems is not entirely achievable because simulating such complex models is difficult, especially when modeling joint frictions and other complex body contact interactions. Furthermore, exact PD computed-torque control typically requires long computing times which makes online real-time control unfeasible without the use of a powerful computing system, which puts a significant constraint on the portability aspect of the manipulator.

Thus, it is desirable to simplify the control law and evaluate whether this simplified model provides accurate control or if a more complex model is required. Following this logic, the control applied to the simulation involves a special case of Eq. 54 for which M(θ)=0 and V(θ,θ)=0 and the addition of an integral of the error term. The proposed control scheme, commonly referred to as the PID Control with Gravity Compensation method, is typically used in practice because it is simpler to implement than the full computed-torque method and yet still provides relatively satisfactory control capabilities. The only required model knowledge is the gravity term, G(θ), which may be readily computed offline or online, either via simulation or from the actual mechanism torque measurements. The control law for this algorithm is:

τ_{C}=K_{P}ε+K_{D}\dot{ε}+K_{I}∫εdt+G(θ) Eq. 57

The control law established in Eq. 57 was therefore used in the simulation. However, in order to implement this algorithm, the gravity effects are first computed.

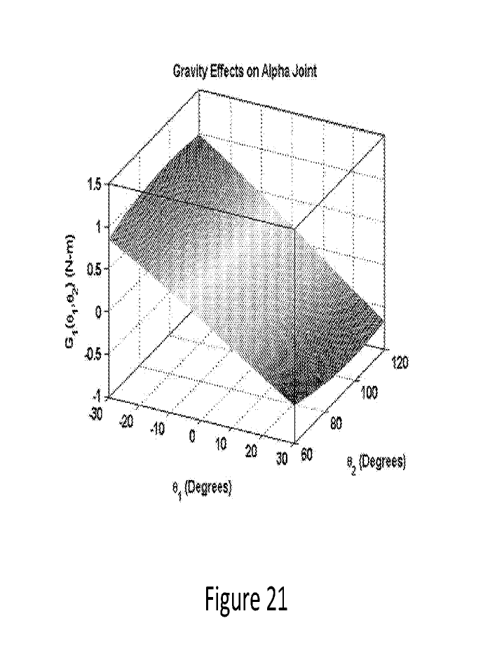

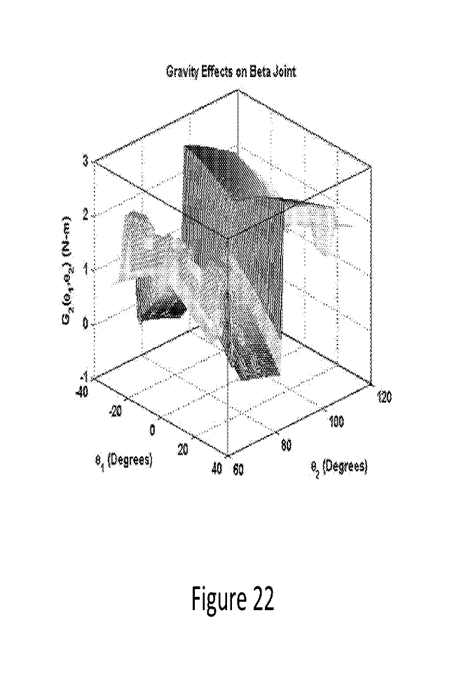

Computing the gravitational effects of the overall mechanism on the α and β joints was achieved by in MSC.ADAMS™ by applying stiff torsional torques to each of the joints and measuring the torque sensed at each spring. The two angles, θ₁ and θ₂ were then varied to span the entire predicted workspace. The characteristic gravity effects on both the α and β joints are displayed in FIG. 21 and FIG. 22, respectively. The plots show a somewhat linear relationship between the gravitational loads on the α Joint, whereas the effects on the β Joint are highly nonlinear. The information presented in the plots was implemented in MSC.ADAMS™ with the use of two 3D splines pre-computed before the simulation begins, one for each term of the gravity function.

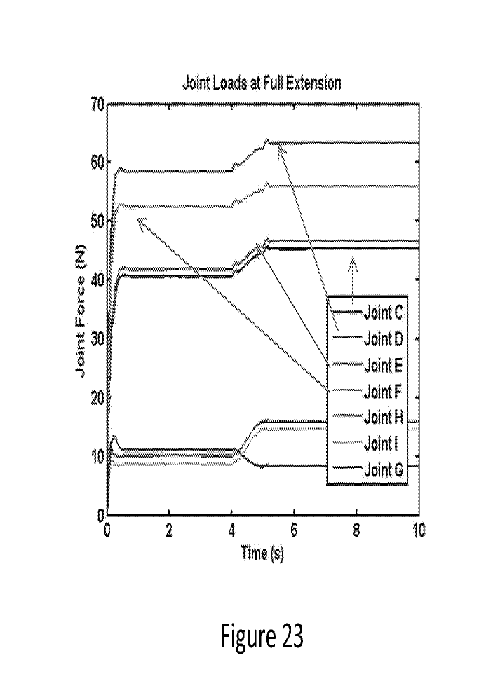

The position control simulations unified all of the parts previously described into a comprehensive simulation capable of orienting the Insertion Module and inserting the implements based on the input vein parameter vectors discussed and displayed in FIG. 20. The results from two sample simulations are presented here, along with the insight gained from each simulation. First, however, the maximum expected joint loads were computed in order to provide the data required for the design of the linkage bearings discussed in further detail below. This simulation was performed by measuring the magnitude of the forces sensed at each link in the Manipulator Module obtained by orienting the mechanism to the configuration that provides the largest loads on the linkage forces. This configuration is the one that extends the mechanism to its longest achievable orientation, which corresponds to an insertion angle of ψ=20° on the β orientation and α=45°. The resulting insertion forces at each joint are plotted in FIG. 23.

Once the maximum joint loads are computed, the next step is to compute the expected actuation torques required to orient the Insertion Module. This information is important to the selection of actuators during the fabrication of the Insertion Mechanism. Thus, two sample simulations are presented below as verification of the performance of the design.

Simulation 1 used the following randomly-generated vein vector, V, as input:

V=[0.5346 −0.6278 0.5658] Eq. 58

This information, yields a target vector, T:

T=[0.2474 −0.9329 0.2618] Eq. 59

and the following orientation angles:

α=15.6779° Eq. 60

β=165.6744° Eq. 61

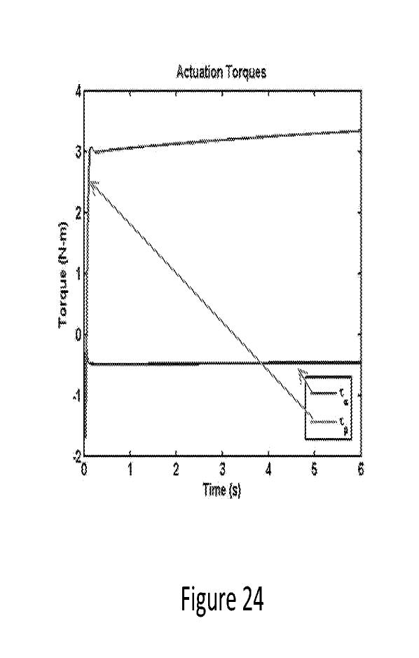

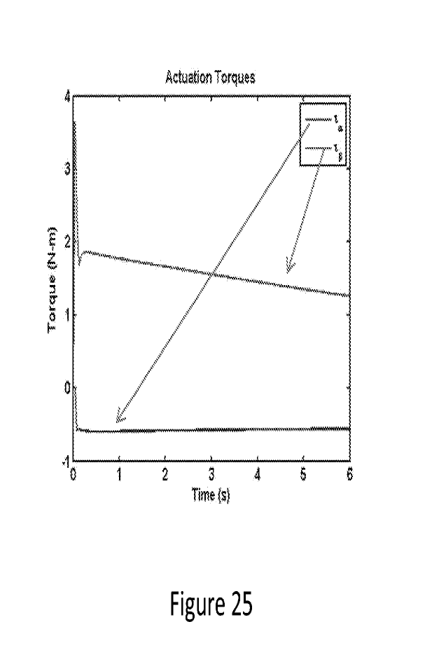

Furthermore, FIG. 24 presents the actuation torques required to achieve this orientation and FIG. 25 shows the orientation error for θ₁ and θ₂. The actuation torques are relatively high, particularly for the actuation of the β joint. This means that the weight of the Insertion Module significantly affects the performance of the manipulator. For the simulations, the mass of Insertion Module was assumed to be 2.5 Kg, which is a conservative estimate that helps provide an upper bound on the actuation torques. Furthermore, the performance of the control algorithm is characterized by the rapid decrease in the orientation error presented in FIG. 25. The rapid decrease in the error may be attributed to the addition of the gravitational terms G(θ), which provides a good estimate of the required actuation torques, given that the actuation speed is relatively slow. For high operational speeds, the inertial and speed-dependent terms of the dynamics of the mechanism become more dominant than the static gravitational terms and thus the performance of this control algorithm decays as operation speed increases.

Simulation 2 used the following randomly-generated vein vector, V, as input:

V=[0.2955 −0.2902 0.3927] Eq. 62

This information, yields a target vector, T:

T=[0.2955 −0.8708 0.3927] Eq. 63

and the following orientation angles:

α=24.2730° Eq. 64

β=162.8140° Eq. 65

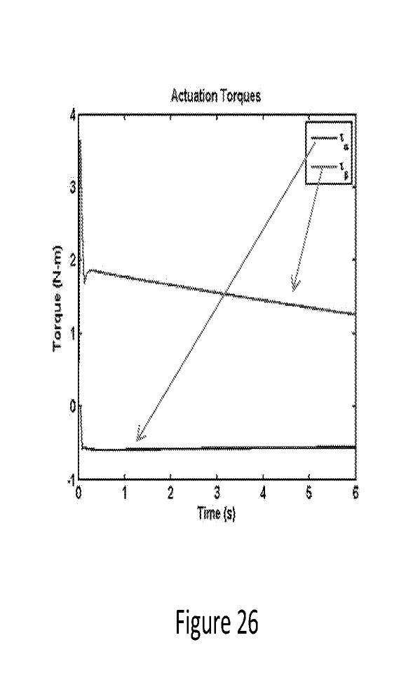

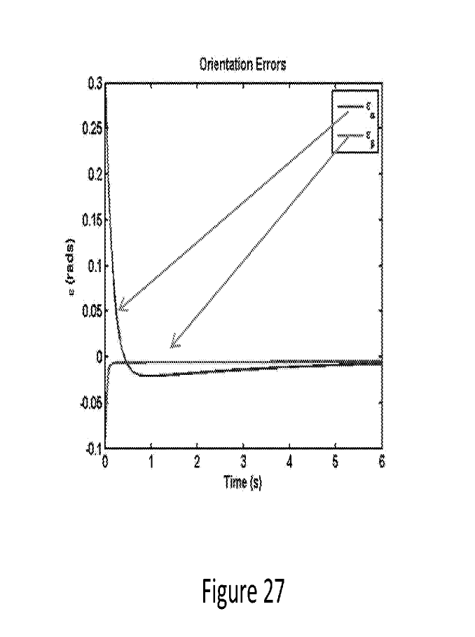

Furthermore, FIG. 26 presents the actuation torques required to achieve this orientation and FIG. 27 shows the orientation errors for θ₁ and θ₂. The results corroborate the results presented in Simulation 1.

### Manipulator Module Fabrication

After validating the performance of the Catheter Insertion Mechanism via the simulations discussed in above, the next step in the design process was to verify the design performance experimentally by developing a fully-functional prototype. This section presents that effort. First, the prototype of the Manipulator Module will be discussed, with particular focus on the challenges encountered during the fabrication and the design choices made, from material to mechanical component selection. The first study discussed consists of measuring the axial forces sensed at the needle base during the insertion of the needle into simulated tissue and vein phantoms. The second involves the measurement of the input and output Manipulator Module orientation angles using the NI Vision toolbox to visually inspect the precision of the linkage.

For the design of the Manipulator Module, the main concern was in the precision of the fabrication of the linkages because a minor error in measurement or any joint misalignment has the potential risk of rendering the whole linkage overconstrained and motionless. Furthermore, the machining of precise angles such as the one required for the links bent at an angle Φ is difficult to achieve without using a CNC Mill or any other type of numerically-controlled machining operation. Thus, to prevent these problems, the linkage parameter values obtained from the simulation in were revised and rounded off to rational values in the English system, with particular focus on rounding off the bent angle, Φ, as close to the nearest whole-degree as possible to ease fabrication complexity, while still maintaining the geometric constraints and the ratios outlined above. Table 7 displays the modified design parameter measurements used in the construction of the prototype.

TABLE 7
Manipulator Linkage Prototype Parameters
Parameter Dimension

| Parameter | Dimension |
|---|---|
| L₁ | 114.30 mm |
| L₂ | 57.15 mm |
| L₃ | 27.65 mm |
| D | 110.90 mm |
| φ | 14° |

Once the geometry of the prototype was defined, the next step was the selection of the material used to build the linkage. Mainly driven by low cost and high structural strength, the selection process was narrowed down to the family of aircraft-strength aluminum alloys. Ultimately, the most commonly used aluminum alloy in aircraft applications was used, Alloy 2024. The material physical properties of this alloy are outlined in Table 8. The material selected for the pins for each rotational joint was ¼ in 303 stainless steel shafts because of the high structural stiffness of stainless steel, which makes it useful to prevent bending at the joints that may cause serious mobility issues to the mechanism. Finally, the last mechanical selection made was for the joint bearings used. As established in the simulation results in discussed above, the peak force sensed at any of the joints was approximately 67 N. To select a suitable bearing, first the bearing load stress, P, should be computed according to the following equation:

$$P = \frac{{F}_{MAX}}{LD} \qquad Eq . 66$$

TABLE 8
Al 2024 Alloy Mechanical Material Properties

| | | |
|---|---|---|
| Physical | Density | $0.1 \frac{lb}{{in}^{3}}$ |
| | Specific Gravity | 2.78 |
| | Melting Point | 950° F. |
| | Temperature Range | −320° to +300° F. |
| Mechanical | Temper | T351 |
| | Hardness | 120 Brinell |
| | Yield Strength | 324 MPa |
| | Ultimate Strength | 469 MPa |
| | Modulus of Elasticity | 73.1 GPa |

where FMAX is the maximum joint load (in lbs), L is the bearing length (in inches), and D is the shaft diameter (in inches). Using the maximum predicted joint load on the linkages measured in the simulations, FMAX ≈ 67N (15.06 lbs), the bearing length, L = 12.7 mm (0.5 in), and the shaft diameter of D = 6.35 mm (0.25 in), the maximum expected bearing load stress is:

$${P}_{MAX} = \frac{15.06}{( 0.5 ) ( 0.25 )} \approx 120 psi \qquad Eq . 67$$

Another major limitation to the selection of a suitable bearing element is that it should have a small profile, in other words, its OD/ID ratio should be as close to unity as possible, without compromising the structural integrity of the bearing. This requirement called for the use of plain flanged sleeve SAE 841 Bronze bearings, which are able to withstand a maximum bearing load of P=2000 psi, require no lubrication, and are simple to mount onto the mechanism.

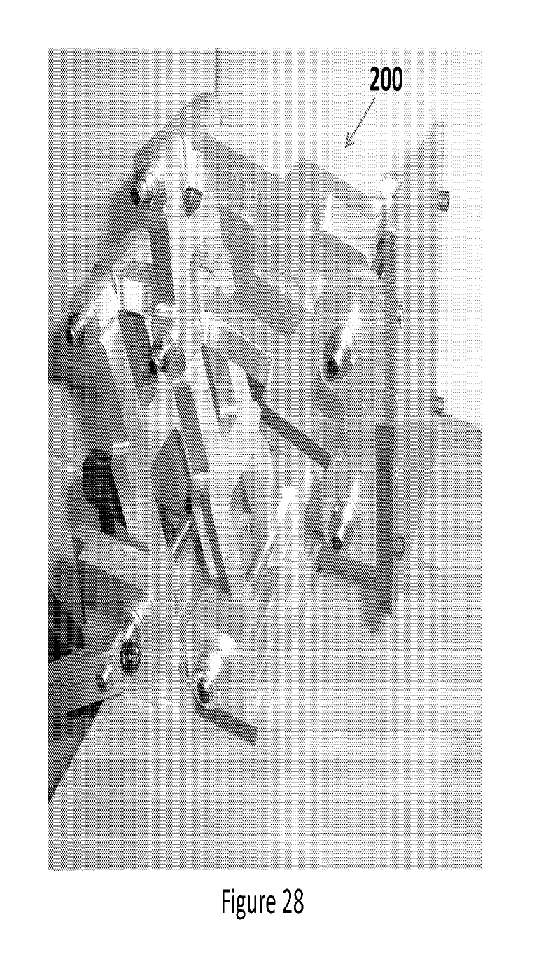

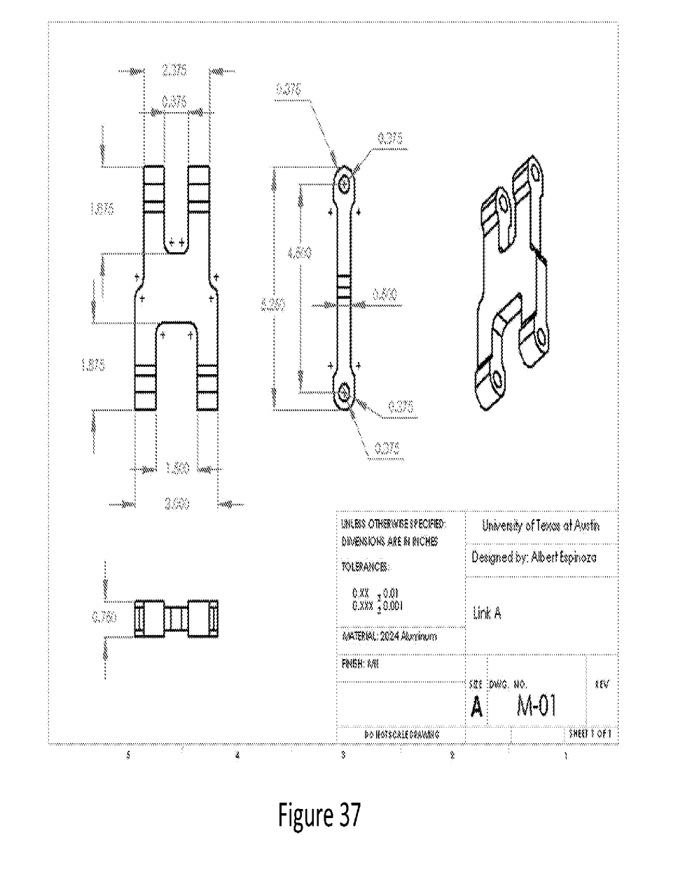

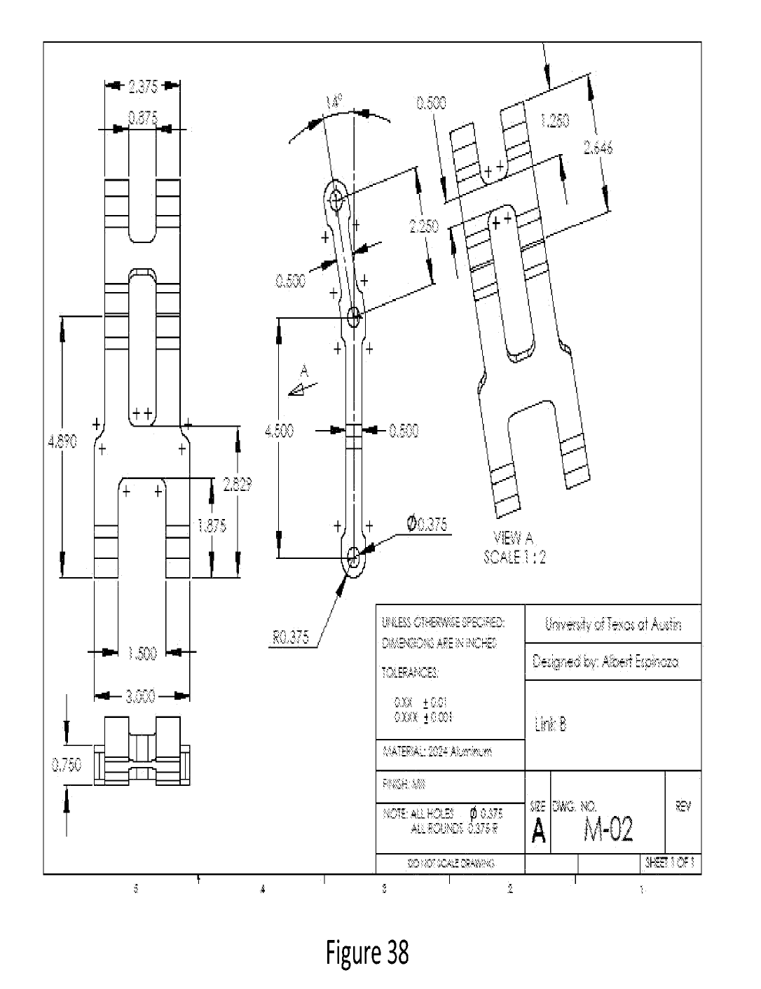

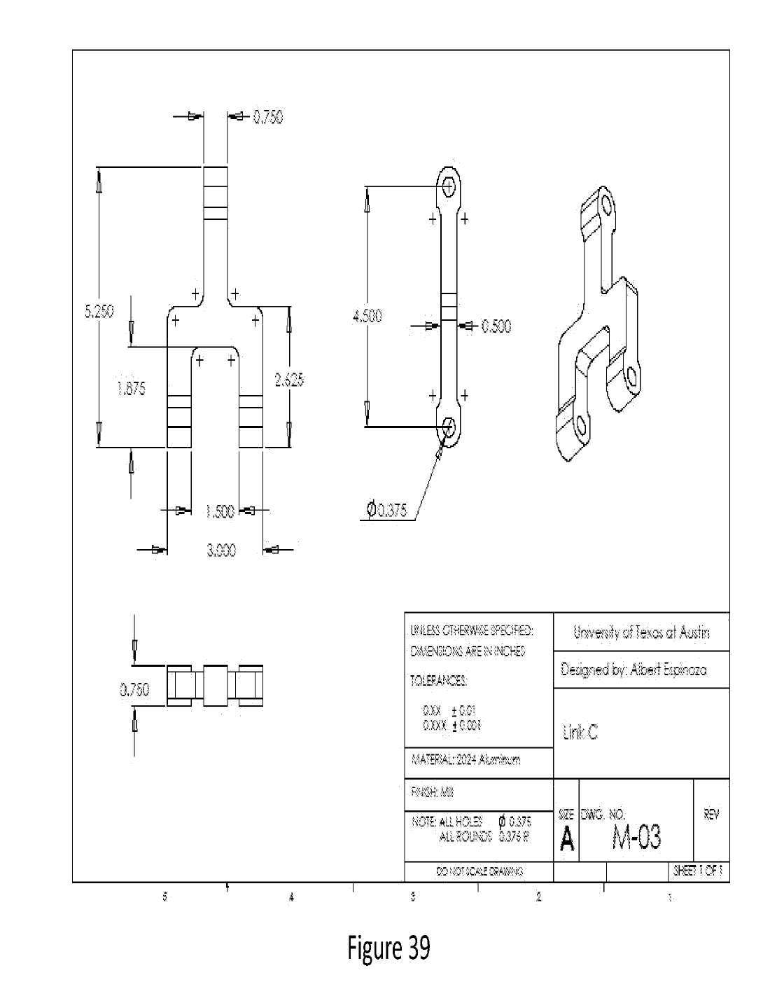

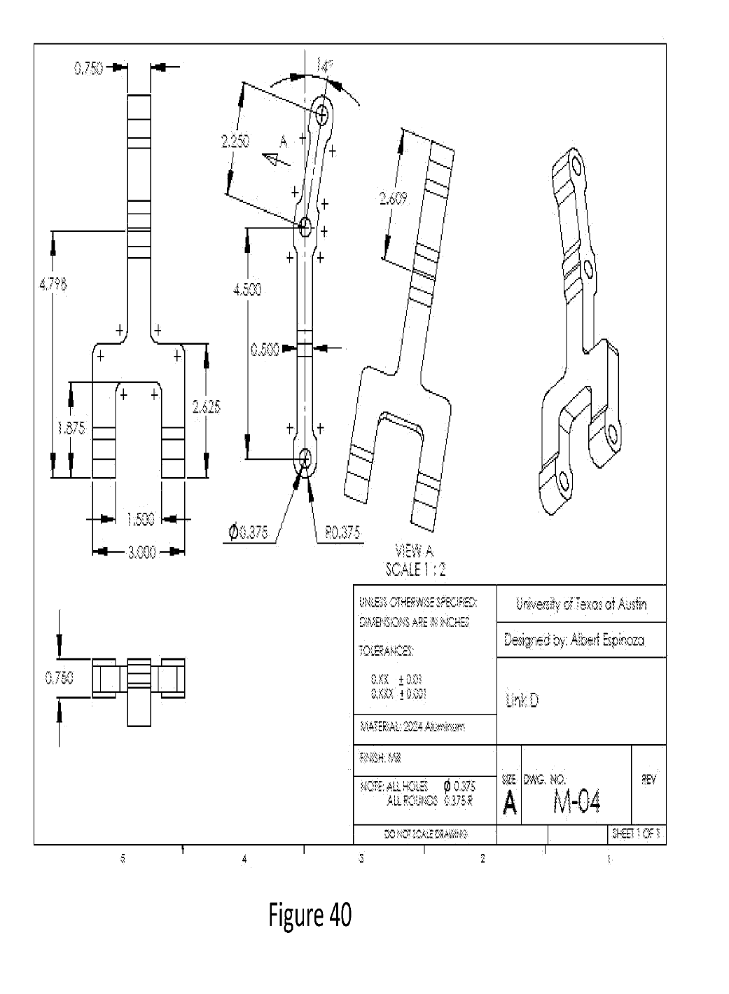

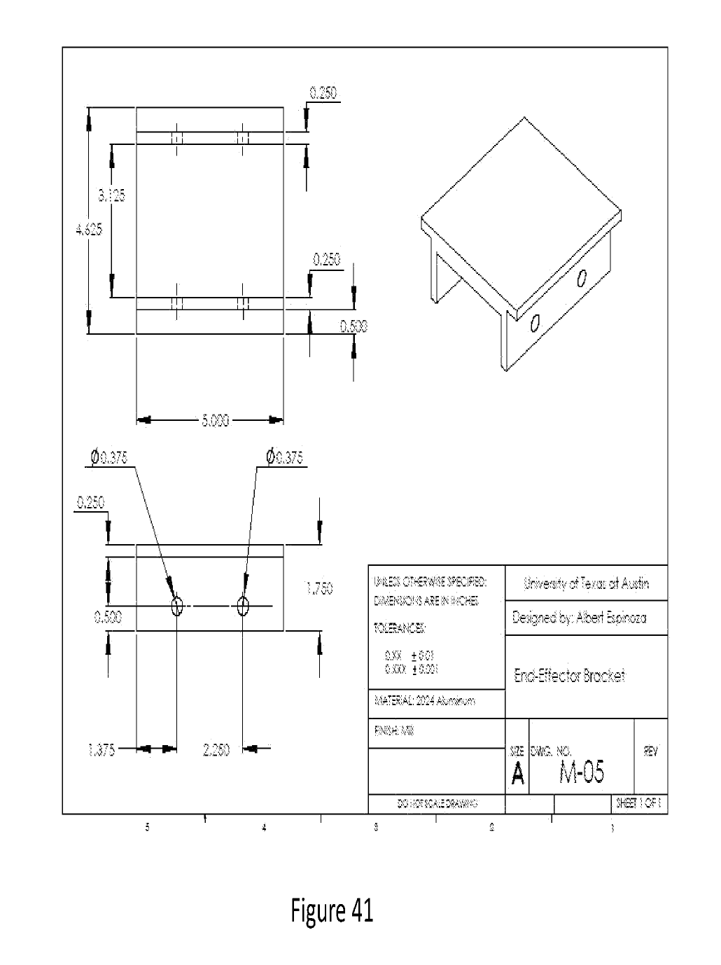

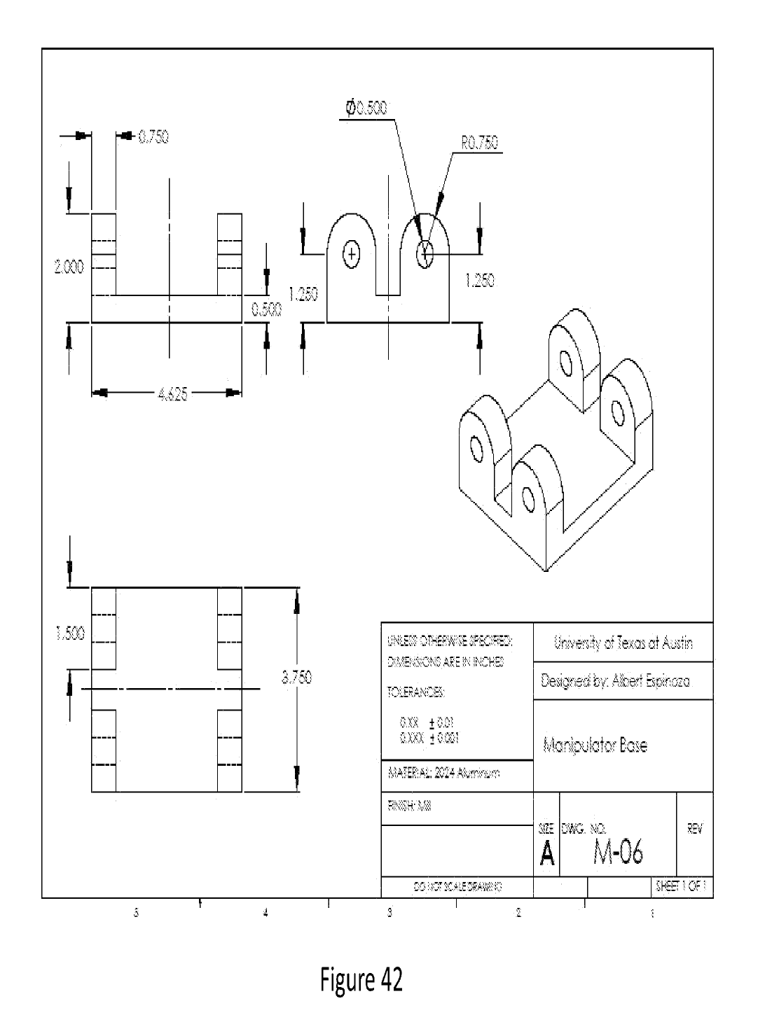

Following the specifications outlined above, the completed Manipulator Module linkage prototype is shown in FIG. 28. For detailed design schematics of each of the Manipulator linkage parts built to date, refer to the mechanical drawings in FIGS. 37-42.

Two main experiments were conducted to validate both the simulation results and the development of the prototype. The first experiment sought out to characterize the axial forces sensed at the tip of a needle as it is inserted into a simulated model of the skin and vein. This experiment was used mainly to gain insight into the mechanics of needle insertions and to use such force model to fit the skin/vein simulation parameters used in the ADAMS simulation. The second experiment involved the use of NI Vision edge detection capabilities to measure the input/output orientation of the Manipulator Module in an attempt to verify the precision of the linkage built and discussed in the previous section.

Throughout the years, there have been numerous studies that have documented the nature of the forces measured during various medical procedures, such as the work conducted by for brachytherapy procedures. Additionally, several studies have measured the needle insertion forces required to puncture through various organs. However, no definitive data exists that presents the forces associated with the insertion of a needle into the skin and vein tissue combination encountered during a femoral vein catheterization. Furthermore, the lack and unavailability of human or animal specimens has led to the search for a suitable substitute tissue phantom that may be used to predict the behavior of skin and vein tissue.

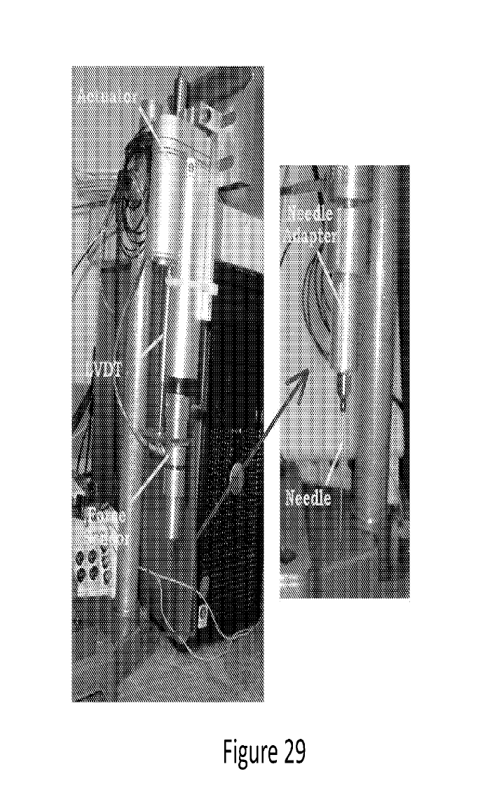

The insertion experiment setup is driven by an Accele™ 12 V DC linear actuator. This actuator provides a maximum insertion stroke of 10.16 cm (4 in.) and up to 489.302 N (110 lb) maximum load with an insertion speed of up to 12.7 mm/s (0.5 in/s). Attached to this actuator is an Omega™ LCFA-10 single axis load cell with a maximum load capacity of 44.48 N (10 lb) used to measure the axial load on the needle during insertion. A 18G needle was attached to the end of the load cell and an Omega™ LD621 LVDT displacement transducer was installed to provide an insertion depth feedback of up to 10.16 cm (4 in.). The data was gathered using National Instruments (NI) LabView™ and a NI PCI-MIO-16E-1 data acquisition card. The experimental setup is depicted in FIG. 29.

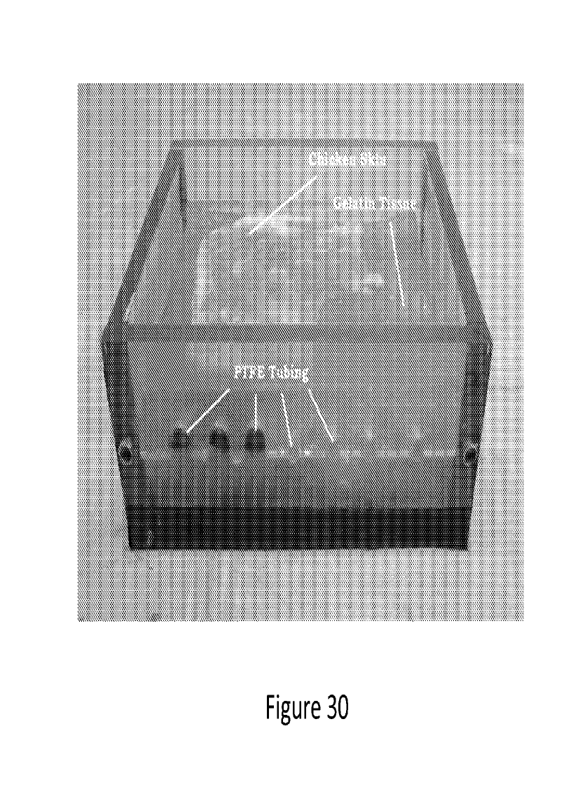

Several phantom materials that simulate the physical characteristics of the skin have been proposed in the literature. Most physical models, however, tend to fall short in performance because the high elasticity of the skin requires complex models that are hard to replicate. Thus, the model for the skin used in these experiments was actual skin tissue cut from a chicken thigh. It is recognized that chicken skin might be dissimilar to human skin tissue in terms of the actual thickness and relative elasticity, but the main objective of these experiments is to capture the general force characteristics of puncturing skin tissue, and in that context, chicken skin provides a suitable substitute. In addition to the skin layer, the inner fatty tissue was modeled with a gel using a 10% by weight concentration solution of unflavored Knox™ gelatin in ionized water. This gel has similar characteristics to the 250 Ordnance™ Type A Gelatin, also manufactured by Kind and Knox Co., which is commonly used by the FBI to simulate soft tissue for ballistics tests. Finally, the model for the vein is a 9.525 mm (⅜ in.), thin-walled PTFE tube manufactured by Zeus, Inc. PTFE was selected because it is commonly used for vascular grafts and should provide comparable characteristics to the femoral vein. The depiction of the skin vein model used in the experiments is shown in FIG. 30.

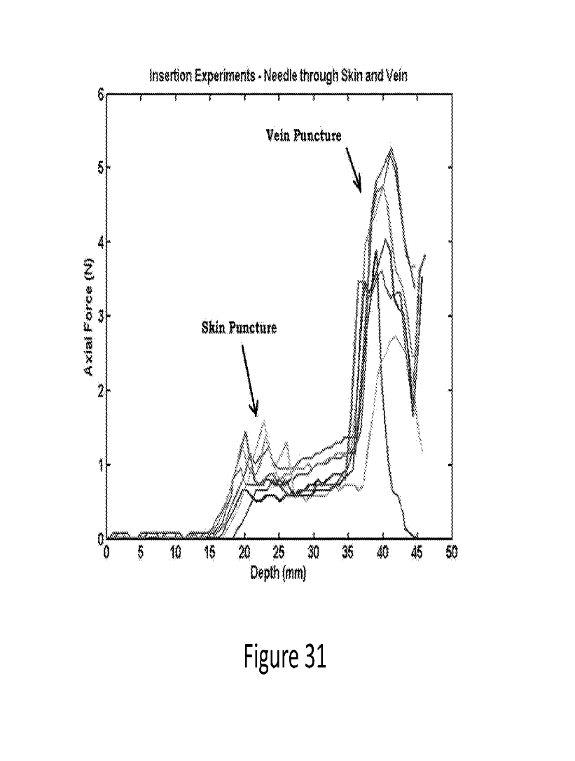

Several insertions were performed using the afore-described setup. The first set of experiments sought to characterize the nature of needle insertions. Particularly, the research points to distinct stages during insertion. The first stage is the Pre-Puncture Stage during which the needle begins to push against the skin and causes it to dimple slightly. This stage is characterized by a slightly increasing force buildup up until the point of puncture. Once the skin is punctured, the axial force slightly decreases as the skin relaxes and slides up the needle shaft, which marks the Relaxation Stage. However, subsequent penetration causes a further rise in the axial force mainly because of frictional and viscous resistance, a stage here referred to as the Viscoelastic Stage. The same behavior is expected as the needle begins to push against the vein. One key feature of great importance to the design of the Insertion Mechanism is the ability to recognize, through force feedback, the impending vein insertion. For instance, the appearance of a noticeable peak in axial force during vein penetration could prove useful in detecting a successful insertion as well as providing information that may be used to guide the needle penetration. However, initial needle puncture tests showed that the PTFE vein model is too stiff to simulate vein tissue. As the plot in FIG. 31 shows, the peak force when the needle tip reaches the vein model is about 4 to 5 N, which is much higher than the force required to puncture the skin/fatty tissue, which is only about 1.4 N. This result is counter-intuitive because the vein is not expected to provide more resistance to penetration than the skin tissue. Thus, subsequent penetrations were performed without the PTFE vein model.

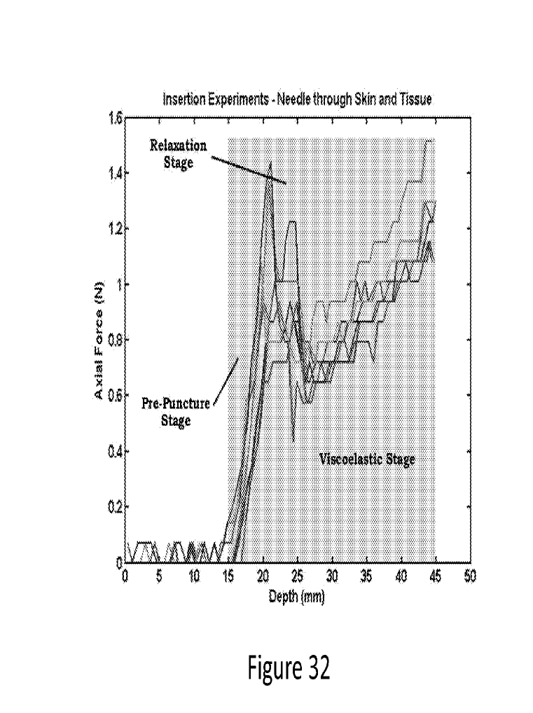

Using just the skin and tissue model, the seven penetration tests shown in FIG. 32 were performed at a constant insertion speed of 4.5 mm/s (0.177 in/s) using only the 18G needle and puncturing different points on the skin/tissue model. As it can be seen from FIG. 32, the vein model does demonstrate the three predicted stages characteristic of needle insertions defined previously. The peak skin-penetration force ranges from roughly 0.9 N up to 1.4 N and the total skin deformation until puncture ranges from about 2 mm to 4 mm. This data is of particular significance to fit the modeling parameters of the simulation discussed above.

In addition to characterizing the insertion forces induced by inserting the needle only, another series of tests were performed to study the effects of inserting implements of various diameters in addition to the needle. These tests will aid in the design of the implement stack that will be simultaneously inserted by the Insertion Module into the insertion region following the sequence discussed above.

Three tests were developed in which different diameter implements were inserted as described below:

1. Test 1. The Needle and a 3.175 mm (⅛ in.) rigid, blunt-tipped dilator tube fitted over the needle were inserted simultaneously. The needle tip, with a length of about 3 mm, sticks out from the tip of the dilator tube.

2. Test 2. Same implements used in Test 1, with the addition of a 4.76 mm ( 3/16 in.) rigid, blunt-tipped dilator tube fitted over the 3.175 mm dilator tube and needle, such that the tip of the 3.175 mm tube sticks out by a distance of 3 mm from the tip of the 4.76 mm dilator.

3. Test 3. Same implements used in Test 2, with the addition of a 6.35 mm (¼ in.) rigid, blunt-tipped dilator tube fitted over the 4.76 mm dilator tube and needle, such that the tip of the 4.76 mm tube sticks out by a distance of 3 mm from the tip of the 6.35 mm dilator.

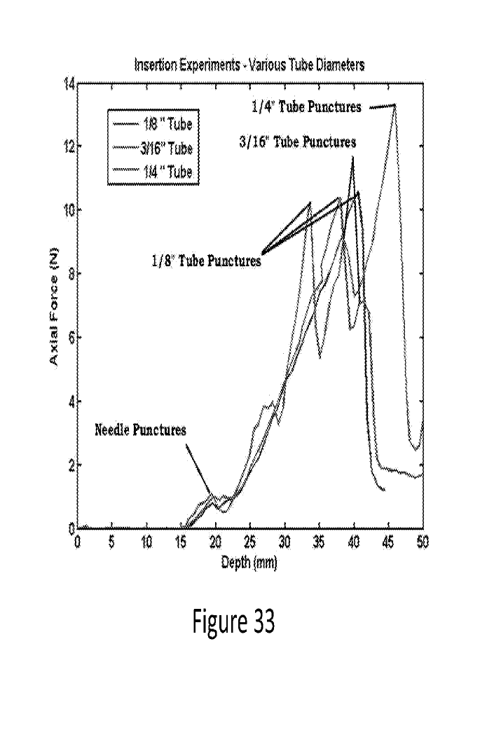

The results from the experiments described above are presented in FIG. 33. Each test is labeled by the size of the largest implement inserted in that particular test.

These tests present the expected increase in the axial forces as the implement diameters increase. However, not only do the insertion forces increase, but the deformation of the skin and the underlying tissue also increase as larger diameter implements are inserted. This behavior is highly undesirable because high tissue deformations cause more trauma to the patient, but most importantly, because large deformations might also cause the target vein to be displaced slightly, which might aggravate the occurrence of back-walling or completely missing the vein. The implements inserted were blunt-tipped to provide the worst case scenario. Thus, careful attention should be given to the design of tapered implements in order to reduce the insertion forces, and with that, reduce the tissue deformation.

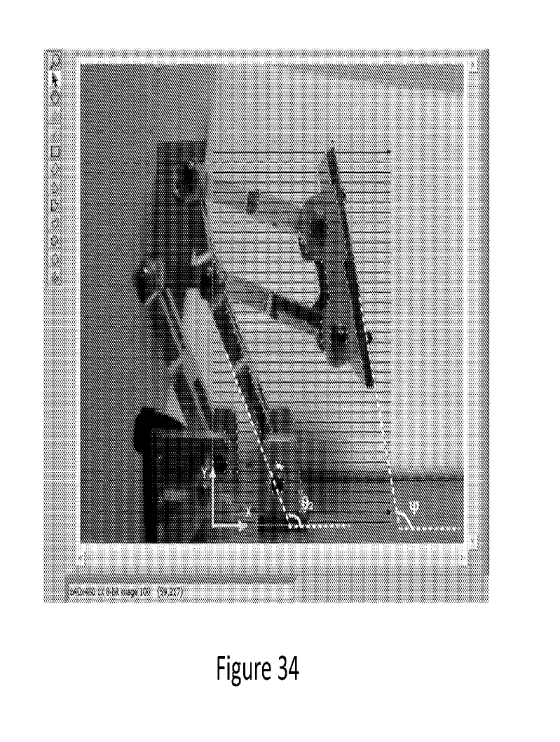

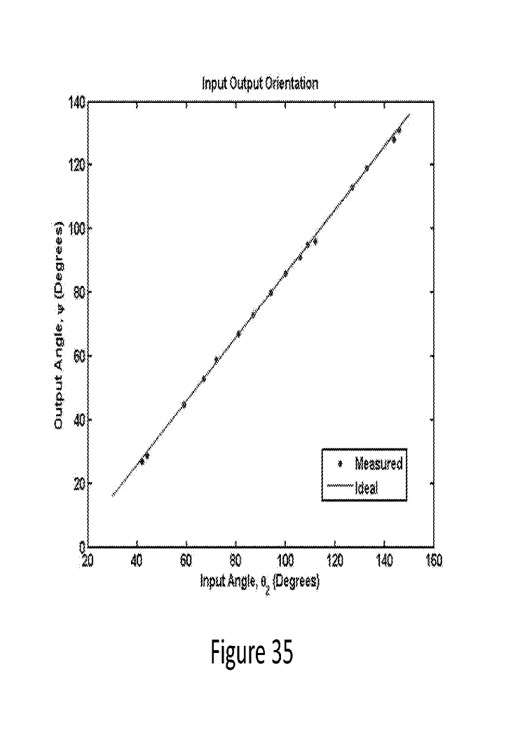

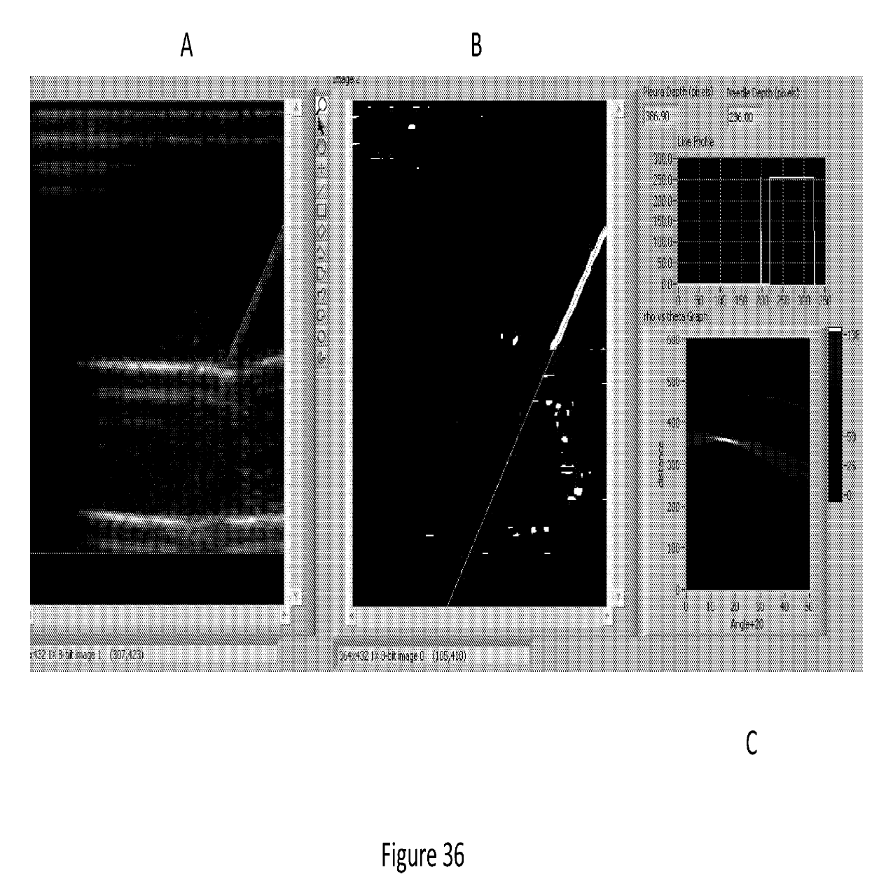

In order to verify that the Manipulator linkage prototype actually produces the predicted output orientation based on a specified input angle, θ₂, the linkage as visually inspected using the computer vision capabilities of NI Vision™ and a NI PCI-1411 Image Acquisition Card. A camera was placed to capture the profile of the linkage mechanism. Red tape markers were mounted on the linkage to provide clear lines that may be readily captured by the image processing LabView Virtual Instrument (VI). A Hough Transform algorithm was used for line detection as implemented in the NI Vision™ toolbox. The algorithm identifies the lines marked with the red tape and outputs each detected line along with the orientation of the line with respect to the search direction (set from left to right). A sample of a line detection frame is shown in FIG. 34. Using the VI, several input-output orientation angle measurements were made. The plot in FIG. 35 shows the input output relationship of the linkage compared to the ideal, expected relationship. The angles are expressed with respect to the positive x-axis as defined in FIG. 34, where counter-clockwise is positive. As the plot demonstrates, the linkage construction does provide close to the expected linear input-output behavior. This means that the fabrication of the linkage is accurate and suitable to use in the orientation of the Insertion Module. The Manipulator Module thus orients the Catheter Insertion Module at the desired orientation after taking into consideration the vein location information. An ultrasound image is shown in FIG. 36 in panel A with the extracted needle position shown by the diagonal line on the right side of the panel. The course of the needle is shown in FIG. 36, panel B. The results of the Hough transform algorithm for line extraction are shown in FIG. 36, panel C.

This effort has resulted in an automated Catheter Insertion Mechanism according to several identified functional requirements obtained from a study of the catheterization process. The design adapted a medical technique that has been proven to be efficient in practice and modified it in order to facilitate the automation of the procedure. Thus, a design was proposed as two independent modules.

The first, denoted as the Catheter Manipulator Module, provides two Remote Center of Motion (RCM) rotational DOFs to orient the second module, the Catheter Insertion Module, arbitrarily in space. Once it is properly oriented, the Insertion Module inserts the implements, ultimately leaving the catheter in place inside the femoral vein. In addition to designing the Insertion Mechanism, a detailed comprehensive simulation was developed to validate the feasibility of the design, as well as to aid in the fabrication of a functional prototype. The simulation may be used to predict the behavior of the mechanism, as well as to make the design and redesign of the mechanism cost and time effective. Additionally, the simulation may also be used to test different control strategies in order to compare their performance and select the best algorithm for this application. Furthermore, in order to ensure the accuracy of the model, an experimental phantom of the skin and vein was developed and the force characteristics of needle insertions into this model were identified and documented to use as baseline data for the model should simulate. In addition to analytical validations, the linkage mechanism was also fabricated and its precision was empirically verified using a vision-based, line detection approach.

The simulation developed and herein discussed shed light into the nature of the mechanics of needle insertions. One of the most significant revelations presented by the simulations was the importance of weight distribution in the design of the Catheter Insertion Module. The simulations demonstrated that even a relatively small increase in the weight of the Insertion Module led to a spike in the loads measured at the joints, as well as a noticeable increase in the actuation torques required to orient the Insertion Module, and most importantly to keep it in place during the insertion of the catheter. The design and material selection should mitigate the high gravitational effects caused by the weight of the Insertion Module. Furthermore, the implement insertion experiments revealed that insertion forces increase greatly when implements of increasingly larger diameters are inserted simultaneously. This aspect may be a significant obstacle to insertion reliability. Thus, tapering the tip of each implement in order to provide a seamless and gradual widening of the insertion wound may be imperative and essential to the design of the Insertion Module.

Although the simulation presented in this document provided valuable insight into the performance of the Catheter Insertion Mechanism, several enhancements may be implemented to provide a deeper understanding of the mechanics of the mechanism. One possible enhancement is the development of a statistical-based analysis that measures the performance of the design under uncertain conditions. In this model, the uncertainty in the model may be quantified and randomized within a predefined “cloud” of uncertainty. For instance, the geometric parameters of the vein, such as diameter and location depth, may be statistically varied and several simulations may be then implemented to quantify the insertion error as a measure of the probability of a successful insertion under this predefined uncertainty. In addition to the simulation, further research efforts may be directed to the development of a fully-functional prototype of the Insertion Module. Design complications that sometimes are unforeseeable from a purely analytical perspective may be evident during building of an Insertion Module prototype. Most importantly, the mechanics of needle insertions into soft tissues, such as the ones encountered during a femoral vein catheterization, should be further studied in order to create mathematical models that may be easily implemented into a robotic mechanism. However, the lack of available needle insertion data into human skin and vascular tissue poses a hurdle in this development. Furthermore, given that visual feedback provides a vital resource to the design of an automated mechanism, the reliability and the characteristics of visual feedback technologies, particularly ultrasound, should be reviewed in order to account for the limitations of visual-feedback techniques.

The system as disclosed herein is well suited for automatically inserting a catheter in a femoral vein of a patient, and can be used to place various tubes into any blood vessel, whether veins or arteries, such as internal jugular and subclavian veins. In either case, both the Manipulator Module and Insertion Module may be mounted on a base or frame such as an X-Y frame, or may be mounted on the arm to position these modules relative to the patient. A suitable X-Y frame is shown in FIG. 34.

Various techniques may be used to form a substantially stationary base, and from that base the Manipulator Module (Positioning Module) may be controlled to desirably position the catheter with respect to the selected point of insertion in the patient. In one embodiment, an arm may be clamped to a side rail of a gurney. In another embodiment, the base may be a chest plate or other contoured plate for positioning over the body of the patient, and then strapped in place to maintain a fixed position of the plate with respect to the patient.

It is a particular feature of the invention that the system includes an Imaging Module, a Manipulator or Positioning Module, and an Insertion Module. In other applications, the Imaging and Positioning Modules may be excluded, and a person may mark the selected point of insertion on the patient, and position the Catheter Insertion Module so that the needle will penetrate that point with a needle, insert the dilator over the needle, retract the needle, and insert the catheter over the dilator, leaving the catheter in place in the blood vessel of the patient.

A preferred embodiment consists of three modules: (1) Manipulation, (2) Insertion and (3) Imaging, along with a central control system. The Manipulation Module is responsible for positioning the Insertion and Imaging Modules in relation to the subject. The Imaging Module acts as the main source of information during the process of insertion, and actively identifies the target area and guides the needle as it is inserted into the body. The insertion module contains the elements used to mechanically cannulate the vessel. Each module has been designed for portability while maintaining a robust structure suitable to survive moderately controlled environments. The system may provide suitable leverage and force to accomplish the desired tasks without any additional source of mechanical power.

Additionally, the system as disclosed herein may also be suitable for use in automatically performing a tracheotomy and tracheostomy (surgical procedures on the neck to open a direct airway through an incision in the trachea). Tracheotomy procedures typically involve the following steps: a curvilinear skin incision along relaxed skin tension lines between sternal notch and cricoid cartilage; a midline vertical incision dividing strap muscles; division of thyroid isthmus between ligatures; elevation of cricoid with cricoid hook; and placement of tracheal incision. An inferior based flap, or Björk flap, (through second and third tracheal rings) is commonly used. The flap is then sutured to the inferior skin margin. Alternatives include a vertical tracheal incision (pediatric) excision of an ellipse of anterior tracheal wall. Insert tracheostomy tube (with concomitant withdrawal of endotracheal tube), inflate cuff, secure with tape around neck or stay sutures. It is also possible to make a simple vertical incision between tracheal rings (typically 2nd and 3rd) for the incision. Rear end flaps may produce more intratracheal granulation tissue at the site of the incisions, making it less favorable to some surgeons.

Percutaneous tracheotomy procedure involves the following steps: curvilinear skin incision along relaxed skin tension lines between sternal notch and cricoid cartilage; midline blunt dissection down to the trachea (optional depending on technique); insertion of 14-gauge plastic cannula and needle with fluid filled syringe attached into trachea. aspiration of air confirms correct placement of the tip in the trachea; removal of needle leaving cannula in place; Insertion of soft tipped guide wire into trachea through cannula; removal of cannula leaving guide wire in place; tracheal dilatation is now undertaken—different techniques do this in different ways.

When available these procedures typically make use of a fiberoptic camera positioned inside the trachea to guide placement of a needle, guidewire and tube via the Seldinger method. In doing so the Percutaneous procedure as described, will often requires two people, one to do the trach placement and one to control the camera. With the presently disclosed system a laser scanner can be used to identify the neck.

The steps of insertion of a trach tube are similar to those for blood vessel. The Manipulator or Positioning Module places the Insertion Module over the neck in the midline. The Imaging module then uses ultrasound to identify the trachea by the tissue-air interface. The rings are easily seen by ultrasound, as is the thyroid gland. The tube (typically an 8 mm tracheostomy tube) is inserted by the Insertion Module at a 45 degree down angle (so that it goes towards the lungs and not the mouth) to enter below the thyroid gland in the midline.

Additionally, the system as disclosed herein may also be suitable for use in automatically performing angiography, insertion of chest drains and central venous catheters, intraosseous cannulation, insertion of percutaneous endoscopic gastrostomy tubes using the push technique, insertion of the leads for an artificial pacemaker or implantable cardioverter-defibrillator, and numerous other interventional medical procedures.

Although specific embodiments of the invention have been described herein in some detail, this has been done solely for the purposes of explaining the various aspects of the invention, and is not intended to limit the scope of the invention as defined in the claims which follow. Those skilled in the art will understand that the embodiment shown and described is exemplary, and various other substitutions, alterations and modifications, including but not limited to those design alternatives specifically discussed herein, may be made in the practice of the invention without departing from its scope.

What is claimed is:

1. A device for automatically inserting a catheter into a blood vessel of a patient, comprising:
an imaging module for identifying a selected point of insertion on the patient;
a manipulator module for positioning the catheter in response to the imaging module at a desired position with respect to the selected point of insertion on the patient; and
a catheter insertion module for inserting a needle into the blood vessel of the patient, inserting a dilator over the needle, retracting the needle, inserting a catheter over the dilator, and retracting the dilator while leaving the catheter in place in the blood vessel of the patient.

2. A device as defined in claim 1, wherein the imaging module includes an ultrasound scanner.

3. A device as defined in claim 2, wherein the imaging module includes a laser scanner.

4. A device as defined in claim 1, further comprising:
the imaging module monitors the position of the patient and the position of the needle while supported on the catheter insertion module.

5. A device as defined in claim 4, wherein the imaging module comprises a linear array ultrasound imaging device.

6. A device as defined in claim 1, further comprising:
a computer for receiving signals from the imaging module and outputting command signals to one of the manipulator module and the catheter insertion module.

7. A device as defined in claim 1, wherein the manipulator module comprises a dual parallelogram linkage mechanism which provides two orthogonal degrees of freedom.

8. A device as defined in claim 7, wherein the linkage mechanism rotates about a first axis which rotates the catheter insertion module about the selected point of insertion; and
the linkage mechanism includes a second axis which rotates the catheter insertion module about a second axis perpendicular to the first axis.

9. A device as defined in claim 1, wherein the catheter insertion module includes a bracket attached to the manipulator module, a powered linear drive for translating the insertion module relative to the manipulator module, and guide rails to guide translation of the insertion module.

10. A device as defined in claim 9, wherein the catheter insertion module includes a locking pin retractor solenoid to release the retraction base to retract both the needle and the dilator.

11. A device as defined in claim 10, further comprising:
a compression spring for retracting the needle and the dilator.

12. A device as defined in claim 1, further comprising:
a pair of gripper arms to hold the catheter in place during insertion.

13. A device for automatically inserting a catheter into a blood vessel of a patient, comprising:
an imaging module for identifying a selected point of insertion on the patient;
a manipulator module for positioning the catheter in response to the imaging module at a desired position with respect to the selected point of insertion on the patient, the manipulator module comprising a dual parallelogram linkage mechanism which provides two orthogonal degrees of freedom; and
a catheter insertion module responsive to the imaging module for inserting the catheter into the blood vessel of the patient.

14. A device as defined in claim 13, wherein the linkage mechanism rotates about a first axis which rotates the catheter insertion module about the selected point of insertion; and
the linkage mechanism includes a second axis which rotates the catheter insertion module about a second axis perpendicular to the first axis.

15. A device as defined in claim 13, further comprising:
a computer for receiving signals from the imaging module and outputting command signals to one of the manipulator module and the catheter insertion module.

16. A device as defined in claim 13, wherein the imaging module includes an ultrasound scanner and a laser scanner.

17. A device as defined in claim 13, wherein the catheter insertion module includes a bracket attached to the manipulator module, a powered linear drive for moving the insertion module relative to the manipulator module, and guide rails to guide translation of the insertion module.

18. A device as defined in claim 13, further comprising:
a pair of gripper arms to hold the catheter in place during insertion.

19. A device as defined in claim 13, wherein:
the catheter insertion module includes a locking pin retractor solenoid to release the retraction base to retract both the needle and the dilator; and
a compression spring for retracting the needle and the dilator.

20. A device for automatically inserting a catheter into a blood vessel of a patient, comprising:
an ultrasound scanner for identifying a selected point of insertion on the patient;
a computer for receiving signals from the ultrasound scanner and outputting command signals to one of a manipulator module and a catheter insertion module;
the manipulator module positioning the catheter in response to the ultrasound scanner at a desired position with respect to the selected point of insertion on the patient; and
the catheter insertion module inserting a needle into the blood vessel of the patient, inserting a dilator over the needle, retracting the needle, inserting a catheter over the dilator, and retracting the dilator while leaving the catheter in place in the blood vessel of the patient.

21. A device as defined in claim 20, further comprising:
the imaging module monitors the position of the needle while supported on the catheter insertion module.

22. A device as defined in claim 20, wherein the catheter insertion module comprises a dual parallelogram linkage mechanism which provides two orthogonal degrees of freedom.

23. A device for automatically inserting a catheter into a blood vessel of a patient comprising:
a catheter insertion module for inserting a needle into the blood vessel of the patient, inserting a dilator over the needle, retracting the needle, inserting a catheter over the dilator, and retracting the dilator while leaving the catheter in place in the blood vessel of the patient.

24. A device as defined in claim 23, wherein the catheter insertion module includes a retractor solenoid to retract both the needle and the dilator.

25. A device as defined in claim 23, further comprising:
a compression spring for retracting the needle and the dilator.

26. A device as defined in claim 23, further comprising:
a pair of gripper arms to hold the cather in place during insertion.

27. A device for automatically inserting a medical implement into a patient, comprising:
an imaging module for identifying a selected point of insertion on the patient;
a manipulator module for positioning the medical implement in response to the imaging module at a desired position with respect to the selected point of insertion on the patient; and
an implement insertion module for inserting the medical implement into the patient.

28. A device as defined in claim 27, wherein the medical implement performs one of a tracheotomy and a tracheostomy in the neck of the patient.

29. A device as defined in claim 27, wherein the imaging module uses ultrasound to identify the trachea by the tissue-air interface.

30. A device as defined in claim 27, further comprising:
a fiber optic camera positioned inside the trachea to guide placement of the medical implement.

31. A device as defined in claim 27, wherein the medical implement is inserted into the chest of the patient.
